(19) United States
(12) Patent Application Publication
(10) Pub. No.: US 2007/0129358 A1
(43) Pub. Date: Jun. 7, 2007

(54) Sbstituted chroman derivatives, processes for their preparation and their use as antiinflammatory agents

(76) Inventors: Norbert Schmees, Berlin (DE); Markus Berger, Berlin (DE); Hartmut Rehwinkel, Berlin (DE); Heike Schaecke, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/414,528

(22) Filed: May 1, 2006

Related U.S. Application Data
(63) Continuation of application No. PCT/EP06/03780, filed on Apr. 13, 2006.
(60) Provisional application No. 60/671,109, filed on Apr. 14, 2005.

(30) Foreign Application Priority Data
Apr. 14, 2005 (DE) 10 2005 017 301.2

(51) Int. Cl. A61K 31/538; A61K 31/502; A61K 31/498; A61K 31/4745; A61K 31/4709; A61K 31/382; A61K 31/405; A61K 31/353; C07D 413/02; C07D 409/02; C07D 405/02; C07D 403/02; C07D 401/02

(52) U.S. Cl. 514/230.5; 544/105; 544/235; 544/237; 544/353; 546/148; 546/160; 549/23; 549/403; 546/118; 548/454; 514/248; 514/249; 514/303; 514/307; 514/314; 514/414; 514/432; 514/456

(57) ABSTRACT

The invention relates to polysubstituted heterocyclic compounds of the general formula (I) 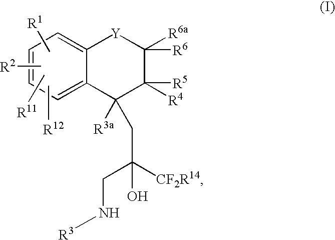 processes for their preparation and their use as anti-inflammatory agents.

This application is a continuation of PCT Application No. ______ filed Apr. 13, 2006 and also claims the benefit of the filing date of U.S. Provisional Application Ser. No. 60/671,109 filed Apr. 14, 2005, both of which are incorporated by reference herein.

The invention relates to substituted chroman derivatives, processes for their preparation and their use as antiinflammatory agents.

Open-chain non-steroidal antiinflammatory agents are known in the art (DE 100 38 639, WO 03/082827 and WO 02/10143). These compounds show experimentally dissociations between antiinflammatory and unwanted metabolic effects and are superior to non-steroidal glucocorticoids described to date or exhibit at least as good an effect.

The present invention provides further non-steroidal antiinflammatory agents.

### BRIEF DESCRIPTION OF THE INVENTION

The present invention relates to compounds of the general formula (I) 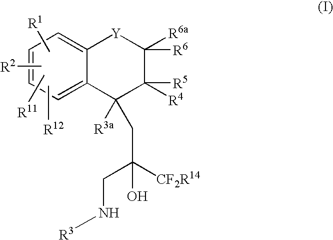
in which

R¹ and R² are independently of one another a hydrogen atom, a hydroxy group, a halogen atom, an optionally substituted (C₁₋₁₀)-alkyl group, a (C₁₋₁₀)-alkoxy group, a (C₁₋₁₀)-alkylthio group, a (C₁₋₅)-perfluoroalkyl group, a cyano group, a nitro group, or an -NR⁹R^{9a} group,
or R¹ and R² together form a group selected from the groups —O—(CH₂)ₙ—O—, —0—(CH₂)ₙ-CH₂—, —O—CH═CH—, —(CH₂)ₙ₊₂—, —NH—(CH₂)ₙ₊₁—, —N(C₁₋₃-alkyl) —(CH₂)ₙ₊₁—and —NH—N═CH—,
where n is 1 or 2, and the terminal oxygen atoms and/or carbon atoms and/or nitrogen atoms are linked to directly adjacent ring carbon atoms,

R¹¹ is a hydrogen atom, a hydroxy group, a halogen atom, a cyano group, an optionally substituted (C₁-C₁₀)-alkyl group, a (C₁-C₁₀ )-alkoxy group, a (C₁-C₁₀)-alkylthio group, or a (C₁-C₅)-perfluoroalkyl group,

R¹² is a hydrogen atom, a hydroxy group, a halogen atom, a cyano group, an optionally substituted (C₁-C₁₀)-alkyl group, or a (C₁-C₁₀)-alkoxy group,

R³ is a (C₁-C₁₀)-alkyl group which is optionally substituted by 1 to 3 hydroxy groups, 1 to 3 halogen atoms, and/or 1 to 3 (C₁-C₅)-alkoxy groups, an optionally substituted (C₃-C₇)-cycloalkyl group, an optionally substituted heterocyclyl group, an optionally substituted aryl group, or a mono- or bicyclic heteroaryl group which is optionally substituted by one or more groups which are selected independently of one another from
(C₁-C₅)-alkyl groups which themselves may optionally be substituted by 1 to 3 hydroxy or 1 to 3 —COOR¹³ groups,
(C₁-C₅) -alkoxy groups, halogen atoms, hydroxy groups, —NR⁹R^{9a} groups, (C₁-C₅)-perfluoroalkyl groups, nitro groups, thiol groups, sulfoxyl groups, sulfonic acid groups, sulfonamide groups, sulfonimine groups, cyano groups or —(CO)—(C₁-C₅)-alkyl groups, and
exo methylene groups and optionally comprises 1 to 4 nitrogen atoms and/or 1 to 2 oxygen atoms and/or 1 to 2 sulfur atoms and/or 1 to 2 keto groups, this group being linked by any position to the nitrogen atom and possibly being optionally hydrogenated at one or more positions,

R^{3a} is a hydrogen atom, a cyano group or an optionally substituted (C₃-C₅)-alkyl group;

R⁴, R⁵, R⁶ and R^{6a} are independently of one another a hydrogen atom, a halogen atom, a hydroxy group, an —NR⁹R^{9a} group, an optionally substituted (C₁-C₁₀)-alkyl group, a (C₁-C₁₀)-alkoxy group or a (C₁-C₁₀)-alkylthio group,

R⁹ and R^{9a} are independently of one another a hydrogen atom, a (C₁-C₅)-alkyl group or a —(CO)—(C₁-C₅)-alkyl group,

R¹⁰ is a (C₁-C₁₀)-alkyl group or a —(CO)—(C₁-C₁₀)-alkyl group,

R¹³ is a hydrogen atom or a (C₁-C₅)-alkyl group,

R¹⁴ is a hydrogen atom, a fluorine atom or a partly or completely fluorinated (C₁-C₅)-alkyl group, and
Y is a methylene group, an oxygen atom, a sulfur atom, an —S(O)ₙ group (where n=1 or 2), an —S(O)(NR¹³) group, an —NH group or an —NR¹⁰ group;
in the form of any stereoisomer or of a mixture of stereoisomers; or as pharmacologically acceptable salt or derivative.

The present invention further relates to processes for preparing compounds of the general formula (I) as described herein.

The present invention further relates to pharmaceutical compositions which include one or more compounds of the general formula (I) in combination with one or more pharmaceutical carriers or excipients.

The present invention additionally relates to the use of the compounds of the general formula (I) for manufacturing pharmaceutical compositions having an antiinflammatory effect.

The present invention further relates to compounds of the general formula (II) 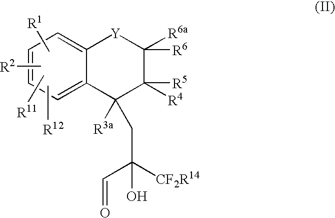
in which the substituents R¹ to R¹⁴ and Y have the abovementioned meanings, and to the use of these compounds for preparing compounds of the general formula (I) as described above.

### DETAILED DESCRIPTION OF THE INVENTION

Definitions

The term halogen atom or halogen means a fluorine, chlorine, bromine or iodine atom. A fluorine, chlorine or bromine atom is preferred.

The alkyl groups mentioned in the definitions of the general formula (I) may be straight-chain or branched and are for example a methyl, ethyl, n-propyl, isopropyl, n-butyl, isobutyl, tert-butyl or n-pentyl, 2,2-dimethylprQpyl, 2-methylbutyl or 3-methylbutyl group, and the hexyl, heptyl, nonyl, decyl group and derivatives thereof branched in any way. Alkyl groups which comprise 1 to 10, 1 to 8 or 1 to 5 carbon atoms are preferred. A methyl or ethyl group is particularly preferred.

The abovementioned alkyl groups may optionally be substituted by 1 to 5, preferably 1 to 3, groups which are selected independently of one another from hydroxy, cyano, nitro, —COOR¹³, (C₃-C₅)-alkoxy groups, halogen atoms, —NR⁹R^{9a}, a partly or completely fluorinated (C₁-C₃)-alkyl group. The alkyl groups may preferably be substituted by 1 to 3 halogen atoms and/or 1 to 3 hydroxy and/or 1 to 3 cyano and/or 1 to 3 -COOR¹³ groups. Fluorine atom, hydroxy, methoxy and/or cyano groups represent a particularly preferred subgroup of substituents.

1 to 3 hydroxy and/or 1 to 3 —COOR¹³ groups are a further particularly preferred group of substituents for the alkyl groups. Hydroxy groups are particularly preferred in this connection.

Examples of a suitable partly or completely fluorinated alkyl group are the following partly or completely fluorinated following groups: fluoromethyl, difluoromethyl, trifluoromethyl, fluoroethyl, 1,1-difluoroethyl, 1,2-difluoroethyl, 1,1,1-trifluoro-ethyl, tetrafluoroethyl, pentafluoroethyl. Of these, the trifluoromethyl or the pentafluoroethyl are preferred. The completely fluorinated group is also called perfluoroalkyl group. The reagents which are optionally employed during the synthesis can be purchased, or the published syntheses of the corresponding reagents belong to the prior art, or published syntheses can be applied analogously.

The alkoxy groups mentioned in the definitions of the general formula (I) may be straight-chain or branched and be for example a methoxy, ethoxy, n-propoxy, isopropoxy, n-butoxy, isobutoxy, tert-butoxy or n-pentoxy, 2,2-dimethylpropoxy, 2-methylbutoxy or 3-methylbutoxy group. C₁-C₅- and C₁-C₃—, C₁-C₈—, and C₁-C₁₀-alkoxy groups are preferred. A methoxy or ethoxy group is particularly preferred.

The alkylthio groups mentioned in the definitions of the general formula (I) may be straight-chain or branched and are for example a methylthio, ethylthio, n-propylthio, isopropylthio, n-butylthio, isobutylthio, tert-butylthio or n-pentylthio, 2,2-dimethylpropylthio, 2-methylbutylthio or 3-methylbutylthio group. C₁-C₅-Alkylthio groups are preferred. A methylthio or ethylthio group is particularly preferred.

The alkoxy and alkylthio groups described above may have on their alkyl groups the same substituents which have been described hereinbefore for the alkyl groups in general. Preferred substituents for alkoxy and alkylthio groups are selected independently of one another from halogen (especially fluorine and/or chlorine), hydroxy and cyano.

The substituent —NR⁹R^{9a} means for example —NH², —NH(CH₃), —N(CH₃)₂, —NH (C₂H₅), —N(C₂H₅)₂, —NH(C₃H₇), —N(C₃H₇)₂, —NH(C₄H₉), —N(C₄H₉)₂, —NH (C₅H₁₁), —N(C₅H₁₁)₂, —NH(CO)CH₃, NH(CO)C₂H₅, —NH(CO)C₃H₇, —NH(CO)C₄H₉, —NH(CO)C₅H₁₁.

The (C₃-C₇)-cycloalkyl group means a saturated cyclic group which is optionally substituted by one or more groups selected from hydroxy groups, halogen atoms, (C₁-C₅) -alkyl groups, (C₁-C₅) -alkoxy groups, —NR⁹R^{9a} groups, —COOR¹³ groups, —CHO, cyano and have 3 to 7 ring carbon atoms such as, for example, cyclopropyl, methyl-cyclopropyl, cyclobutyl, methylcyclobutyl, cyclopentyl, methylcyclopentyl, cyclohexyl, methylcyclohexyl, cycloheptyl, methylcycloheptyl.

An alkylidene or exo alkylidene group means a group having 1 to 10 carbon atoms which is bonded via an exo double bond to the system (ring or chain). (C₁-C₅)- and (C₁-C₃)-alkylidene is preferred, and exo methylene is particularly preferred.

The heterocyclyl group is a cyclic, non-aromatic group comprising one or more heteroatoms and may be for example pyrrolidine, imidazolidine, pyrazolidine, piperidine. Perhydroquinoline and perhydroisoquinoline are also included in the heterocyclyl groups of the invention.

Examples of suitable substituents for heterocyclyl and heteroaryl groups are substituents from the following group: optionally substituted C₁-C₅-alkyl groups, hydroxy, (C₁C₅)-alkoxy, —NR⁹R^{9a}, halogen, cyano, —COOR¹³, —CHO. The substituents may optionally also be bonded to the nitrogen atom of the heterocyclyl or heteroaryl group; N-oxides are also included in the definition.

Aryl groups in the context of the invention are aromatic or partly aromatic carbocyclic groups having 6 to 14 carbon atoms which have one ring, such as, for example, phenyl or phenylene, or a plurality of fused rings, such as, for example, naphthyl or anthranyl. Examples which may be mentioned are phenyl, naphthyl, tetralinyl, anthranyl, indanyl, and indenyl. The optionally substituted phenyl group and the naphthyl group are preferred.

The aryl groups may be substituted at any suitable position leading to a stable compound by one or more radicals from the group of hydroxy, halogen, C₁-C₅-alkyl which is optionally substituted by 1 to 3 hydroxy groups or COOR¹³ groups, or C₁-C₅-alkoxy, cyano, —CF₃ and nitro.

The aryl groups may be partly hydrogenated and then, in addition or as alternative to the substituents detailed above, also carry keto and/or exo alkylidene. Partly hydrogenated phenyl means for example cyclohexadienyl, cyclohexenyl, cyclohexyl. A partly hydrogenated substituted naphthalene system is for example 1-tetralone or 2-tetralone.

The mono- or bicyclic heteroaryl group may optionally comprise 1 to 9 groups selected from nitrogen atoms, oxygen atoms, sulfur atoms or keto groups, of which a maximum of 4 nitrogen atoms, a maximum of 2 oxygen atoms, a maximum of 2 sulfur atoms and/or a maximum of 2 keto groups may be present. Every subcombination of these groups is possible. The heteroaryl group may be hydrogenated at one or more positions.

Monocyclic heteroaryl groups may be for example pyridine, pyrazine, pyrimidine, pyridazine, triazine, azaindolizine, 2H- and 4H-pyran, 2H- and 4H-thiopyran, furan, thiophene, 1H- and 4H-pyrazole, 1H- and 2H-pyrrole, oxazole, thiazole, furazan, 1H- and 4H-imidazole, isoxazole, isothiazole, oxadiazole, triazole, tetrazole, thiadiazole.

Bicyclic heteroaryl groups may be for example phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzo[b]thienyl, benzo[c]thienyl, pyrazolo[1,5-a]-pyridyl, benzimidazolyl, dihydroisoquinolinyl, dihydroquinolinyl, benzoxazinonyl, phthalazinonyl, dihydrophthalazinonyl, quinolinyl, isoquinolinyl, quinolonyl, isoquinolonyl, quinazolinyl, quinoxalinyl, cinnolinyl, phthalazinyl, dihydrophthalazinyl, 1,7- or 1,8-naphthyridinyl, coumarinyl, isocoumarinyl, indolizinyl, isobenzofuranyl, azaindolyl, azaisoindolyl, furanopyridyl, furanopyrimidinyl, furanopyrazinyl, furanopyridazinyl, dihydrobenzofuranyl, dihydrofuranopyridyl, dihydrofuranopyrimidinyl, dihydrofuranopyrazinyl, dihydrofuranopyridazinyl, dihydrobenzofuranyl group.

If the heteroaryl groups are partly or completely hydrogenated, the present invention includes compounds of the general formula (I) in which R³ is tetrahydro-pyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylideneaminyl, chromanyl, isochromanyl, thiochromanyl, decahydroquinolinyl, tetrahydroquinolinyl, dihydro-quinolinyl, 5,6,7,8-tetrahydro-1-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz-[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-lH-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, 2,2-dihydro-1H-pyrrolo[2,3-b]pyridin-3-onyl.

The mono- or bicyclic heteroaryl group may optionally be substituted by one or more substituents selected from C₁-C₅-alkyl groups which are optionally substituted by 1 to 3 hydroxy groups or 1 to 3 —COOR¹³ groups, or C₁-C₅-alkoxy groups, halogen atoms, and/or exo methylene groups. The substituents may, if possible, optionally also be bonded directly to the heteroatom (e.g. to the nitrogen atom). The present invention also includes N-oxides.

Suitable hydroxy protective groups which are necessary where appropriate are all conventional hydroxy protective groups known to the skilled worker, in particular silyl ethers or esters of organic C₁-C₁₀ acids, C₁-C₅ ethers, benzyl ethers or benzyl esters. Conventional hydroxy protective groups are described in detail in T. W. Greene, P. G. M. Wuts “Protective Groups in Organic Synthesis”, 2nd edition, John Wiley & Sons, 1991). The protective groups are preferably alkyl-, aryl- or mixed alkylaryl-substituted silyl groups, e.g. the trimethylsilyl (TMS), triethylsilyl (TES), tert-butyldimethylsilyl (TBDMS), tert-butyldiphenylsilyl (TBDPS) or triisopropylsilyl groups (TIPS) or other customary hydroxy protective groups (e.g. methoxymethyl, methoxyethoxymethyl, ethoxyethyl, tetrahydrofuranyl, tetrahydropyranyl groups).

The compounds of the invention of the general formula (I) may, owing to the presence of centers of asymmetry, exist as stereoisomers. The present invention relates to all possible diastereoisomers both as racemates and in enantiopure form. The term stereoisomers also includes all possible diastereoisomers and regioisomers and tautomers (e.g. keto-enol tautomers) in which the stereoisomers of the invention may exist, and to which the invention therefore likewise relates.

The compounds of the invention may also be in the form of salts with pharmacologically acceptable anions, for example in the form of the hydrochloride, sulfate, nitrate, phosphate, pivalate, maleate, fumarate, tartrate, benzoate, mesylate, citrate or succinate.

The invention also includes pharmacologically suitable derivatives or prodrugs of the compounds of the general formula (I). Derivatives or prodrugs refers for example to esters, ethers or amides of the compounds of the general formula (I) or other compounds which metabolize in the body to compounds of the general formula (I).

Suitable compounds are listed for example in Hans Bundgaard (Editor), Design of Prodrugs, Elsevier, Amsterdam 1985.

Preferred Embodiments

A subgroup of compounds of the invention of the general formula (I) are those compounds in which R¹ and R² are independently of one another a hydrogen atom, a hydroxy group, a halogen atom, an optionally substituted. (C₁-C₁₀)-alkyl group, a (C₁-C₁₀)-alkoxy group, a (C₁-₁₀)-alkylthio group, a (C₁-C₅)-perfluoroalkyl group, a cyano group, a nitro group, or an —NR⁹R^{9a} group.

A preferred group of compounds of the general formula (I) are those compounds in which Y is an oxygen atom, a sulfur atom or a methylene group.

A further preferred group of compounds of the general formula (I) are those compounds in which R³ is an optionally substituted aryl or heteroaryl group. A more preferred group of compounds of the general formula (I) are those compounds in which R³ is an optionally substituted aryl or heteroaryl group which is selected from the group consisting of naphthyl, phthalidyl, isoindolyl, dihydroindolyl, dihydroisoindolyl, dihydroisoquinolinyl, thiophthalidyl, benzofuranyl, benzoxazinonyl, phthalazinonyl, quinolinyl, isoquinolinyl, quinolonyl, isoquinolonyl, chromanyl, isochromanyl, indazolyl, benzothiazolyl, quinazolinyl, quinoxalinyl, cinnolinyl, phthalazinyl, 1,7- or 1,8-naphthyridinyl, pyrazolo[1,5-alpyridyl, dihydro-indolonyl, dihydroisoindolonyl, benzimidazole or indolyl group.

A further preferred group of compounds of the general formula (I) are those compounds in which R^{3a} is a hydrogen atom or a (C₁-C₅)-alkyl group.

A further preferred group of compounds of the general formula (I) are those compounds in which R⁴, R⁵, R⁶, and R^{6a} are independently of one another a hydrogen atom, a halogen atom or an optionally substituted (C₁-C₁₀)-alkyl group.

A further preferred group of compounds of the general formula (I) are those compounds in which R¹ and R² are independently of one another a hydrogen atom, a halogen atom or an optionally substituted (C₁-C₁₀)-alkyl group.

A further preferred group of compounds of the general formula (I) are those compounds in which the substituents R¹¹ and R¹² are in each case a hydrogen atom.

A further preferred group of compounds of the general formula (I) are those compounds in which the substituent R¹⁴ is a fluorine atom or a trifluoromethyl group.

A particularly. preferred group of compounds of the general formula (I) are those compounds in which R¹, R², R⁴, R⁶, and R^{6a} are independently of one another a hydrogen atom, a halogen atom or an optionally substituted (C₁-C₁₀)-alkyl group, Y is an oxygen atom, a sulfur atom or a methylene group, R^{3a} is a hydrogen atom or a (C₁-C₅)-alkyl group, R¹¹ and R² are in each case a hydrogen atom, R¹⁴ is a fluorine atom or a trifluoromethyl group, and R³ is an optionally substituted aryl or heteroaryl group which is selected from the group consisting of naphthyl, phthalidyl, isoindolyl, dihydroindolyl, dihydroisoindolyl, dihydroisoquinolinyl, thiophthalidyl, benzofuranyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, pyrazolo[1,5-a]pyridyl, dihydroindolonyl, chromanyl, isochromanyl, dihydroisoindolonyl, benzimidazole or indolyl group.

Every further possible combination of the abovementioned subgroups and of substituents indicated as preferred with their general and/or specific meanings is likewise to be regarded as encompassed by the present invention.

Biological Activity

The antiinflammatory effect of the compounds of the general formula (I) is tested in an animal experiment by testing in the croton oil-induced inflammation in the rat and mouse (J. Exp. Med. (1995), 182, 99-108). For this purpose, croton oil is applied in ethanolic solution topically to the ears of the animals. The test substances are likewise administered topically or systemically at the same time as or two hours before the croton oil. After 16-24 hours, the ear weight is measured as a measure of the inflammatory edema, the peroxidase activity is measured as a measure of the migration in of granulocytes and the elastase activity is measured as a measure of the migration in of neutrophilic granulocytes. In this test, the compounds of the general formula (I) inhibit the three abovementioned parameters of inflammation both after topical and after systemic administration.

The binding of the substances to the glucocorticoid receptor (GR) and further steroid hormone receptors (mineral corticoid receptor (MR), progesterone receptor (PR) and androgen receptor (AR)) is examined with the aid of recombinantly prepared receptors. Cytosol preparations of Sf9 cells which had been infected with recombinant baculoviruses which code for the GR are employed for the binding studies. Compared with the reference substance [³H]-dexamethasdne, the substances show a high affinity for the GR. Thus, an IC₅₀(GR)=20 nM and IC₅₀(PR) >1 μM was measured for the compound of example 1 and an IC₅₀(GR)=30 nM and IC₅₀(PR) >1 μM was measured for the compound of example 2.

The essential molecular mechanism for the antiinflammatory effect of glucocorticoids is regarded as the GR-mediated inhibition of the transcription of cytokines, adhesion molecules, enzymes and other pro-inflammatory factors. This inhibition is brought about by an interaction of the GR with other transcription factors, e.g. AP-1 and NF-kappa-B (for review, see Cato ACB and Wade E, BioEssays 18, 371-378 1996).

The compounds of the invention of the general formula (I) inhibit the secretion, induced by lipopolysaccharide (LPS), of the cytokine IL-8 in the human THP-1 monocyte cell line. The concentration of the cytokines was determined in the supernatant using a commercially available ELISA kit.

One of the commonest unwanted effects of a glucocorticoid therapy is the so-called “steroid diabetes” [cf. Hatz, HJ, Glucocorticoide: Immunologische Grundlagen, Pharmakologie und Therapierichtlinien, Wissenschaftliche Verlagsgesellschaft mbH, Stuttgart, 1998]. The cause of this is stimulation of gluconeogenesis in the liver by induction of the enzymes responsible therefor and by free amino acids resulting from the breakdown of proteins (catabolic effect of the glucocorticoida). A key enzyme in catabolic metabolism in the liver is tyrosine aminotransferase (TAT). The activity of this enzyme can be determined by photometry on liver homogenates and represents a good measure of the unwanted metabolic effects of glucocorticoids. To measure the TAT induction, the animals are sacrificed 8 hours. after administration of the test substances, the livers are removed, and the TAT activity in the homogenate is measured. In this test, the compounds of the general formula (I) induce, in doses in which they have antiinflammatory activity, tyrosine aminotransferase to only a small extent or not at all.

Medical Indications

Owing to their antiinflammatory and additional anti-allergic, immunosuppressive and antiproliferative effect, the compounds of the invention of the general formula (I) can be used as medicaments for the treatment or prophylaxis of the following pathological states in patients, especially mammals and preferably humans: in this connection, the term “DISORDER” stands for the following indications:
(i) pulmonary disorders associated with inflammatory, allergic and/or proliferative processes:
   chronic obstructive lung disorders of any origin, especially bronchial asthma
   bronchitis of varying origin
   all types of restrictive lung disorders, especially allergic alveolitis,
   all types of pulmonary edema, especially toxic pulmonary edema
   sarcoidoses and granulomatoses, especially Boeck's disease
(ii) rheumatic disorders/autoimmune diseases/joint disorders associated with inflammatory, allergic and/or proliferative processes:
   all types of rheumatic disorders, especially rheumatoid arthritis, acute rheumatic fever polymyalgia rheumatica
   reactive arthritis
   inflammatory soft tissue disorders of other origin
   arthritic symptoms associated with degenerative joint disorders (arthroses)
   traumatic arthritides
   collagenoses of any origin, e.g. systemic lupus erythematosus, scleroderma, polymyositis, dermatomyositis, Sjögren's syndrome, Still's syndrome, Felty's syndrome
(iii) allergies associated with inflammatory and/or proliferative processes:
   all types of allergic reactions, e.g. angioedema, hayfever, insect bite, allergic reactions to drugs, blood derivatives, contrast agents etc., anaphylactic shock, urticaria, contact dermatitis
(iv) vessel inflammations (vasculitides)
   polyarteritis nodosa, temporal arteritis, erythema nodosum
(v) dermatological disorders associated with inflammatory, allergic and/or proliferative processes:
   atopic dermatitis (especially in children)
   psoriasis
   pityriasis rubra pilaris
   erythematous disorders induced by various noxae, e.g. radiation, chemicals, burns, etc.
   bullous dermatoses
   lichenoid disorders
   pruritus (e.g. of allergic origin)
   seborrheic eczema
   rosacea
   pemphigus vulgaris
   erythema multiforme exudativum
   balanitis
   vulvitis
   hair loss such as alopecia areata
   cutaneous T-cell lymphomas
(vi) renal disorders associated with inflammatory, allergic and/or proliferative processes:
   nephrotic syndrome
   all nephritides
(vii) liver disorders associated with inflammatory, allergic and/or proliferative processes:
   acute liver cell necrosis
   acute hepatitis of varying origin, e.g. viral, toxic, drug-induced
   chronic aggressive and/or chronic intermittent hepatitis
(viii) gastrointestinal disorders associated with inflammatory, allergic and/or proliferative processes:
   regional enteritis (Crohn's disease)
   ulcerative colitis
   gastritis
   reflux esophagitis
   gastroenteritides of other origin, e.g. indigenous sprue
(ix) proctological disorders associated with inflammatory, allergic and/or proliferative processes:
   anal eczema
   fissures
   hemorrhoids
   idiopathic proctitis
(x) ocular disorders associated with inflammatory, allergic and/or proliferative processes;
   allergic keratitis, uveitis, iritis,
   conjunctivitis
   blepharitis
   optic neuritis
   chorioditis
   sympathetic ophthalmia
(xi) ear-nose-throat disorders associated with inflammatory, allergic and/or proliferative processes:
   allergic rhinitis, hayfever
   otitis externa, e.g. caused by contact exema, infection etc.
   otitis media
(xii) neurological disorders associated with inflammatory, allergic and/or proliferative processes;
   cerebral edema, especially tumor-related cerebral edema
   multiple sclerosis
   acute encephalomyelitis
   meningitis
   various types of seizures, e.g. infantile spasms
(xiii) blood disorders associated with inflammatory, allergic and/or proliferative processes:
   acquired hemolytic anemia
   idiopathic thrombocytopenia
(xiv) neoplastic disorders associated with inflammatory, allergic and/or proliferative processes:
   acute lymphatic leukemia
   malignant lymphomas
   lymphogranulomatoses
   lymphosarcomas
   extensive metastases, especially associated with breast, bronchial and prostate carcinomas
(xv) endocrine disorders associated with inflammatory, allergic and/or proliferative processes
   endocrine orbitopathy
   thyrotoxic crisis
   de Quervain's thyroiditis
   Hashimoto's thyroiditis
   Basedow's disease
(xvi) organ and tissue transplantations, graft-versus-hose disease
(xvii) severe states of shock, e.g. anaphylactic shock, systemic inflammatory response syndrome (SIRS)
(xviii) emesis associated with inflammatory, allergic and/or proliferative processes:
   e.g. in combination with a 5-HT3 antagonist in cytostic-related vomiting.
(xix) pain of inflammatory origin, e.g. lumbago
(xx) replacement therapy for:
   congenital primary adrenal insufficiency, e.g. congenital adrenogenital syndrome
   acquired primary adrenal insufficiency, e.g. Addison's disease, autoimmune adrenalitis, post-infection, tumors, metastases etc.
   congenital secondary adrenal insufficiency, e.g. congenital hypopituitarism
   acquired secondary adrenal insufficiency, e.g. post-infection, tumors etc.

Medicaments comprising stereoisomers of the general formula I show a particular efficacy for the following disorders:
1. lung disorders
2. rheumatic disorders/autoimmune diseases
3. dermatological disorders
4. degenerative joint disorders
5. vessel inflammations
6. graft versus host disease
7. severe states of shock
8. emesis associated with inflammatory, allergic and/or proliferative processes
9. inflammation-related pain.

In addition, the compounds of the invention of the general formula (I) can be employed for the therapy and prophylaxis of further pathological states which are not mentioned above but for which synthetic glucocorticoids are currently used (concerning this, see Hatz, H J, Glucocorticoide: Immunologische Grundlagen, Pharmakologie und Therapierichtlinien, Wissenschafliche Verlagsgesellachaft mbH, Stuttgart, 1998).

All the aforementioned indications are described in detail in Hatz, HJ, Glucocorticoide: Immunologische Grundlagen, Pharmakologie und Therapierichtlinien, Wissenschafliche Verlagsgesellachaft mbH, Stuttgart, 1998).

The suitable dose for a therapeutic effect in the abovementioned pathological states varies and depends for example on the potency of the compound of the general formula (I), the patient (e.g. height, weight, gender, etc.), the mode of administration and the nature and severity of the conditions to be treated, and the use as prophylactic or therapeutic agent.

The invention relates to the use of the claimed compounds for manufacturing a pharmaceutical composition.

The invention further provides:
(i) the use of one of the compounds of the invention of the general formula (I) or mixture thereof for manufacturing a pharmaceutical composition for the treatment or prevention of inflammatory processes, and especially for the treatment of a DISORDER (as defined above);
(ii) a method for the treatment or prevention of inflammatory processes, especially for the treatment of a DISORDER (as defined above), which method includes administration of a pharmaceutically effective amount of a compound of the general formula (I), where the amount alleviates or suppresses the disease or the symptoms, and where the compound is given to a patient, preferably a mammal, in particular a human, requiring such a treatment;
(iii) a pharmaceutical composition having an antiinflammatory effect, in particular for the treatment of a DISORDER (as defined above), where the composition includes one of the compounds of the invention or mixture thereof and, where appropriate, at least one pharmaceutical excipient and/or carrier.

Satisfactory results are generally to be expected in animals when the daily doses include a range from 1 μg to 100 000 μg of the compound of the invention per kg of body weight. For larger mammals, for example humans, a recommended daily dose is in the range from 1 μg to 100 000 μg per kg of body weight. A dose of 10 to 30 000 μg per kg of body weight is preferred, and a dose of 10 to 10 000 μg per kg of body weight is more preferred. This dose is for example expediently administered more than once a day. For the treatment of acute shock (e.g. anaphylactic shock) it is possible to give single doses which are distinctly higher than the abovementioned doses.

The pharmaceutical products based on the novel compounds are formulated in a manner known per se by processing the active ingredient with the carrier substances, fillers, substances influencing disintegration, binders, humectants, lubricants, absorbents, diluents, masking flavors, colorants etc. which are in use in pharmaceutical technology, and converting into the desired administration form. Reference should be made in this connection to Remington's Pharmaceutical Science, 15th ed. Mack Publishing Company, East Pennsylvania (1980).

Particularly suitable for oral administration are tablets, coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injections or suspensions and corresponding depot preparations can be used for intramuscular injection.

The novel compounds can be used for rectal administration in the form of suppositories, capsules, solutions (e.g. in the form of enemas) and ointments both for systemic and for local therapy.

The novel compounds can be used in the form of aerosols and inhalations for pulmonary administration thereof.

For local use on eyes, the external auditory canal, middle ear, nasal cavity and paranasal sinuses, the novel compounds can be used as drops, ointments, tinctures and gels in appropriate pharmaceutical preparations.

Formulations possible for topical application are gels, ointments, greasy ointments, creams, pastes, dusting powders, suspensions, emulsions and solutions. The dosage of the compounds of the general formula (I) in these preparations should be 0.01% -20% in order to achieve an adequate pharmacological effect.

The invention likewise includes the compounds of the invention of the general formula (I) as therapeutic active ingredient. The invention further includes the compounds of the invention of the general formula (I) as therapeutic active ingredient together with one or more pharmaceutically suitable and acceptable excipients and/or carriers.

The compounds of the invention of the general formula (I) can also where appropriate be formulated and/or administered in combination with further active ingredients. therapies or combined compositions in which a compounds of the general formula (I) or a pharmaceutically acceptable salt thereof, or a pharmaceutical composition comprising a compound of the general formula (I) or a pharmaceutically acceptable salt thereof, is administered either simultaneously (where appropriate in the same composition) or successively together with one or more medicaments for the treatment of one of the pathological states mentioned above. For the treatment of rheumatoid arthritis, osteoarthritis, COPD (chronic obstructive pulmonary disorder), asthma or allergic rhinitis, for example, it is possible to combine a compound of the general formula (I) of the present invention with one or more medicaments for the treatment of such a condition. Where such a combination is administered by inhalation, the medicament to be combined can be selected from the following list:
a PDE4 inhibitor including an inhibitor of the PDE4D isoform;
a selective β.sub2.adrenoceptor agonist such as, for example, metaproterenol, isoproterenol, isoprenaline, albuterol, salbutamol, formoterol, salmeterol, terbutaline, orciprenaline, bitolterol mesylate, pirbuterol or indacaterol;
a muscarine receptor antagonist (for example an M1, M2 or M3 antagonist, such as, for example, a selective M3 antagonist) such as, for example, ipratropium bromide, tiotropium bromide, oxitropium bromide, pirenzepine or telenzepine;
a modulator of chemokine receptor function (such as, for example, a CCR1 receptor antagonist); or
an inhibitor of p38 kinase function.

For another aspect of the present invention, such a combination with a compound of the general formula (I) or a pharmaceutically acceptable salt thereof is employed for the treatment of COPD, asthma or allergic rhinitis and can be administered by inhalation or orally in combination with xanthine (such as, for example, aminophylline or theophylline), which can likewise be administered by inhalation or orally.

Preparation Processes

The compounds of the invention of the general formula (I) can be obtained in various ways. The preparation processes described below likewise form a part of the present invention.

Unless indicated otherwise, the substituents used in the process descriptions below have the same meaning as above in the section “Brief description of the invention”, including the definitions stated in the section “Detailed description of the invention”.

In general, compounds of the general formula (I) are prepared by reacting aldehydes of the general formula (II) with amines of the general formula R³-NH₂ and subsequent reduction of the imines obtained thereby. The reaction sequence is summarized in scheme 1 below.

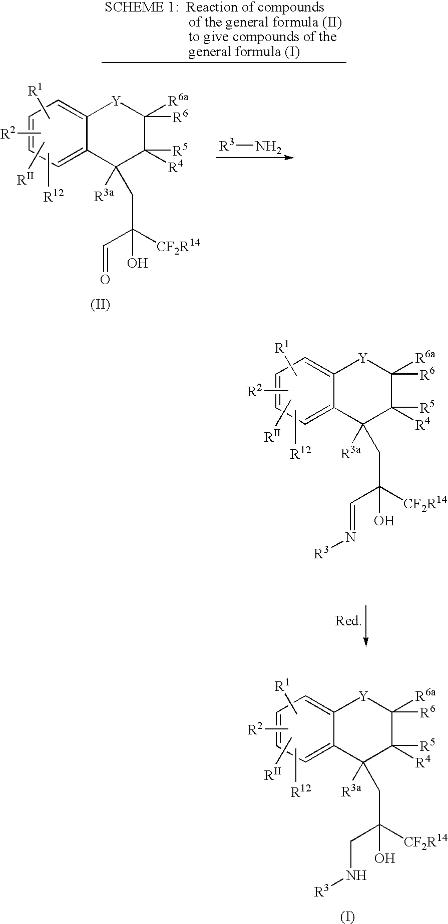

The compounds of the general formula (II) used in this sequence can be obtained by various processes according to the invention.

Process A: Preparation of compounds of the general formula (II) from compounds of the general formula (IX) via intermediate (IIIA)

In the first step of process A, compounds of the general formula (Ix) in which R is preferably a C₁-C₅-alkyl group are reacted to give compounds of the general formula (VIII) (scheme 2). Compounds of the general formula (IX) are known in the art and can be prepared by processes known to the skilled worker.

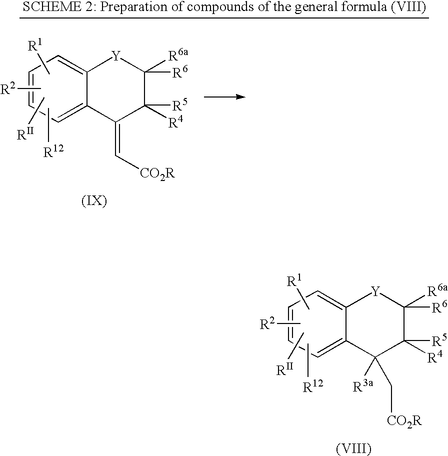

If R^{3a} in the compounds of the general formula (VIII) is a hydrogen atom, the reaction takes place by reduction, e.g. by catalytic hydrogenation of the compound (IX) with elemental hydrogen over suitable metal catalysts. Compounds of the general formula (VIII) in which R^{3a} is a cyano or alkyl group are obtainable by reacting compounds of the general formula (IX) with cyanide ions or organometallic alkyl compounds (e.g. organocopper compounds). These processes are known in the art.

In the next step, the carboxylic esters of the general formula (VIII) obtained in this way are reduced with a suitable reducing agent (e.g. lithium aluminum hydride) by processes known in the art to alcohols of the general formula (VII) (scheme 3).

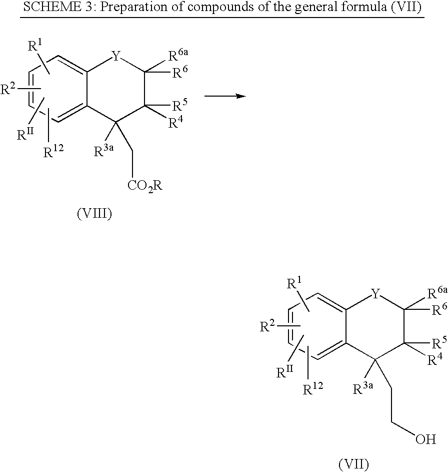

The aldehydes of the general formula (VI) are obtained by careful oxidation of the alcohols of the general formula (VII) by processes known in the art (scheme 4):

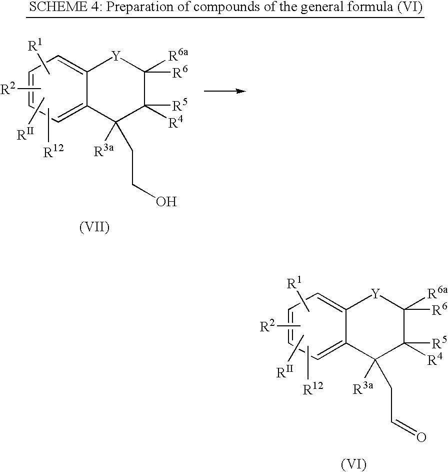

The aldehydes of the general formula (VI) can be reacted with suitable organometallic reagents M-CF₂R¹⁴, thus making it possible to introduce the group —CF₂R¹⁴ into the molecule (scheme 5), where M is an electrophilic leaving group. Examples of suitable organometallic reagents are compounds of the general formula CₙF₂ₙ₊₁-Si(CH₃)₃ in the presence of a catalyst. Suitable as catalyst in this process step are fluoride salts or basic compounds such as alkali metal carbonates (J. Am. Chem. Soc. 1989, 111, 393). Alternatively, organolithium or -magnesium compounds can also be used to introduce the group —CF₂R¹⁴. In this case, the alcohols of the general formula (V) are obtained.

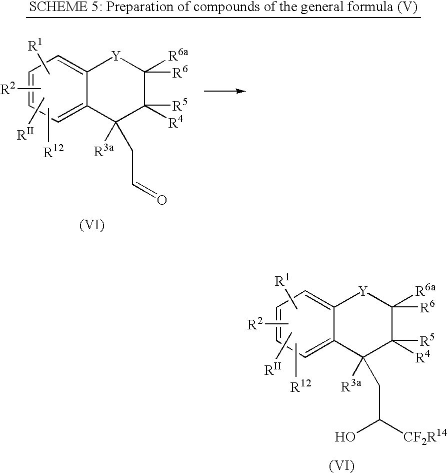

Ketones of the general formula (IV) can be obtained by oxidation of the alcohols of the general formula (V) using processes known in the art (e.g. with Dess-Martin periodinane as oxidizing agent) (scheme 6)

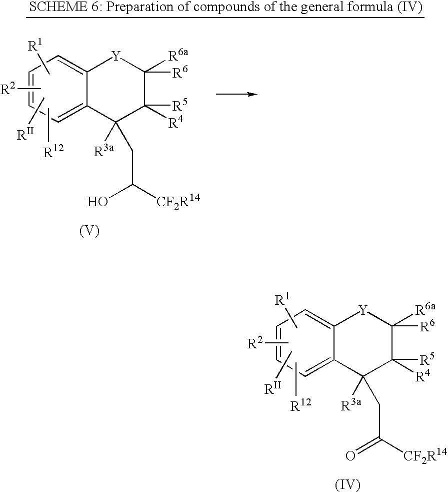

The ketones of the general formula (IV) are then converted into the corresponding cyanohydrins of the general formula (IIIA) (scheme 7). Reagents suitable for this purpose are, for example, potassium, sodium or copper cyanide, or else trimethylsilyl cyanide. According to the reagent employed, R in the general formula (IIIA) is a hydrogen atom or a trimethylsilyl group.

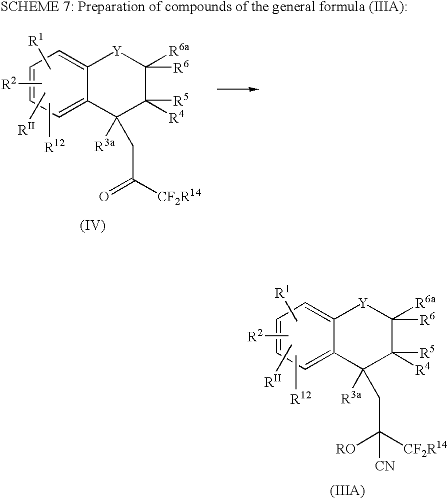

The cyanohydrins of the general formula (IIIA) are then, where appropriate after removal of the trimethyl-silyl group—converted with a suitable reducing agent (e.g. diisobutylaluminum hydride) into the aldehydes of the general formula (II) (scheme 8). 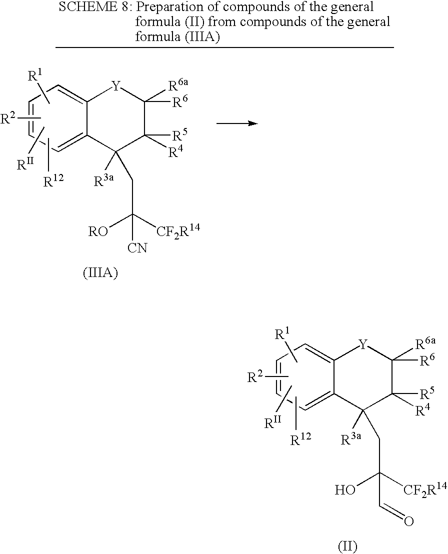
Process B: Preparation of compounds of the general formula (II) from compounds of the general formula (IX) via intermediates (IIIB)

Synthesis of the precursor compounds of the general formulae (VIII), (VII), (VI), (V) and (IV) proceeds in process B as in process A.

The aldehyde of the general formula (IV) is then converted with a suitable organometallic reagent (e.g. a Grignard compound such as, for example, vinylmagnesium bromide) into an unsaturated alcohol of the general formula (IIIB) (scheme 9).

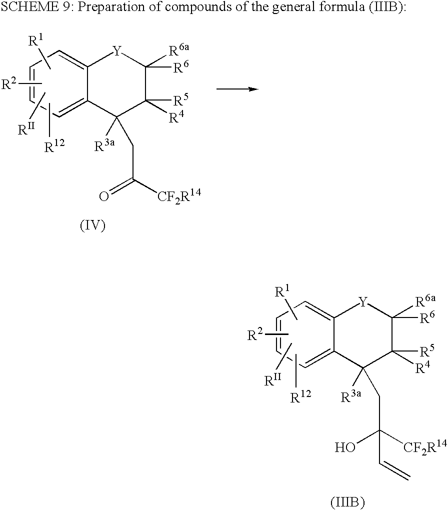

Compounds of the general formula (II) can be obtained by oxidizing the double bond in the compounds of the general formula (IIIB) (e.g. by ozone or by transition metal oxides such as, for example, osmium tetraoxide with subsequent cleavage using a suitable oxidizing agent such as, for example, sodium periodate) (scheme 10). 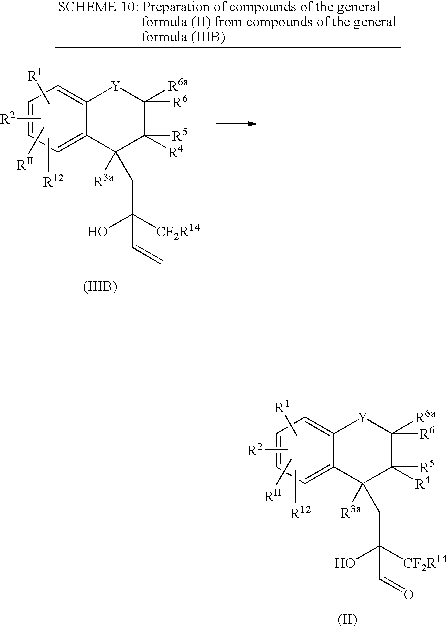
Process C: Preparation of compounds of the general formula (II) from compounds of the general formula (XIII)

Compounds of the general formula (II) are prepared from compounds of the general formula (XIII) known in the art by firstly reacting the latter with α-keto esters of the general formula R-¹⁴CF₂—C(═O) —CO₂R (where R is preferably a C₁-C₅-alkyl group) with catalysis by—optionally chiral—Lewis acids in an ene reaction to give compounds of the general formula (XII) (scheme 11).

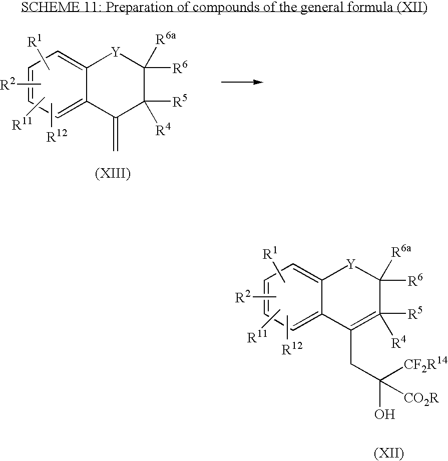

The diol of the general formula (XI) can be obtained from the compound of the general formula (XII) by reducing the ester function by processes known in the art (e.g. with lithium aluminum hydride) (scheme 12).

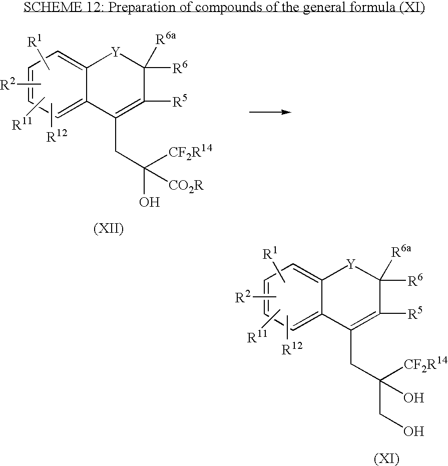

This diol (XI) can be converted into a compound of the general formula (X) by reducing the double bond (e.g. by catalytic hydrogenation with elemental hydrogen on a suitable metal catalyst) (scheme 13).

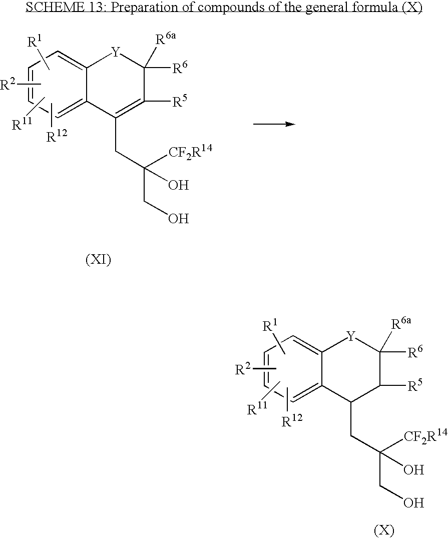

Aldehydes of the general formula (II) in which the substituents R^{3a} and R⁴ in the compounds of the general formulae (I) and (II) have the meaning of a hydrogen atom can be obtained from these diols of the general formula (X) by oxidizing the terminal hydroxy group (scheme 14).

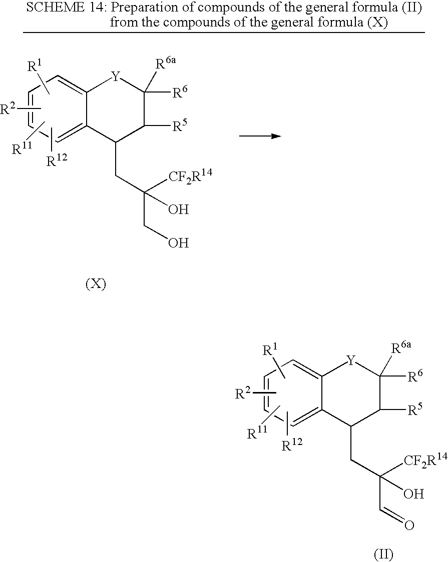

As an alternative to the reaction sequence indicated above for process C, esters of the general formula (XII) can also be reduced to the corresponding aldehydes, which can be reacted directly with the primary amines of the general formula R³—NH₂. Subsequently, the isolated double bond present in the right-hand ring of the compound is hydrogenated with suitable reducing agents. This alternative sequence also results in compounds of the general formula (I) in which the substituents R^{3a} and R⁴ have the meaning of a hydrogen atom.

### EXAMPLES

Synthesis 1: Preparation of compounds of the general formula (VII)

A 2-(Chroman-4-yl)ethanol:

2.27 g of ethyl (2-chrornan-4-yl)acetate (J. Med. Chem. 44, (2001), pp. 1085-1098) are stirred with 800 mg of lithium aluminum hydride in 50 ml of THF at 0° C. for 2 hours. Saturated ammonium chloride solution and ethyl acetate are added to the mixture, which is then filtered through kieselguhr. The aqueous phase is extracted with ethyl acetate, and the combined organic phases are washed with brine and dried with sodium sulfate. 1.9 g of 2-(chroman-4-yl)ethanol are obtained as crude yield.

¹H NMR (CDCl₃): δ 1.72-1.90 (m, 2H), 2.0-2.2 (m, 2H), 2.96-3.09 (m, 1H)₁ 3.80 (t, 2H), 4.13-4.25 (m, 2H), 6.79 (d, 1H), 6.87 (t, 1H), 7.10 (t, 1H), 7.14 (d, 1H).

B. 2-(4-Methylchroman-4-yl)ethanol:

11.5 ml of methylmagnesium chloride solution [3.3M in THF] are slowly added to 7.3 g of ethyl E/Z-(chroman-4-ylidene)acetate (J. Med. Chem. 44, (2001), pp. 1085-1098), 100 mg of copper (I) chloride and 5.1 ml of chlorotrimethylsilane in 55 ml of THF at 0° C. in such a way that the temperature always remains below 5° C. The mixture is stirred at 0° C. for one hour and at room temperature for 10 hours. Saturated ammonium chloride solution is added to the mixture, which is then extracted with ether. The combined organic phases are washed with brine, dried with sodium sulfate and concentrated in vacuo. Chromatography on silica gel (hexane/ethyl acetate 100;0->90:10) results in ethyl 2-(4-methylchroman-4-yl)acetate as crude product. This is mixed in 50 ml of THF at 0° C. with 1 g of lithium aluminum hydride and stirred at 0° C. for 1.5 hour. The mixture is cautiously added to sat ammonium chloride solution and diluted with ethyl acetate. It is filtered through kieselguhr, and the aqueous phase is extracted with ethyl acetate. The combined organic phases are washed with brine, dried with sodium sulfate and concentrated in vacuo. Chromatography on silica gel (hexane/ethyl acetate 100:0->60:40) results in 1.2 g of 2-(4-methylchroman-4-yl)ethanol as colorless oil.

¹NMR (CDCl₃): δ=1.36 (s, 3H), 1.74 (ddd, 1H) , 1.91-2.11 (m, 3H), 3.57-3.75 (m, 2H), 4.10-4.28 (m, 2H), 6.79 (d, lH), 6.89 (t, lH), 7.08 (t, 1H), 7.22 (d, 1H).

C. The following can likewise be prepared by the described processes:
2-(6-methylchroman-4-yl)ethanol
2-(7-methylchroman-4-yl)ethanol
2-(6-fluorochroman-4-yl)ethanol
2-(6-methoxychroman-4-yl)ethanol
2-(7-methoxychroman-4-yl)ethanol
2-(1,2,3,4-tetrahydronaphthalen-1-yl)ethanol
2-(2-methyl-1,2,3,4-tetrahydronaphthalen-1-yl)ethanol
2-(4-methyl-1,2,3,4-tetrahydronaphthalen-1-yl)ethanol
2-(5-methoxy-1,2,3,4-tetrahydronaphthalen-1-yl)ethanol
2-(5,7-dimethyl-1,2,3,4-tetrahydronaphthalen-1-yl)-ethanol
2-(6-methoxy-1,2,3,4-tetrahydronaphthalen-1-yl)ethanol
2-(6-fluoro-1,2,3,4-tetrahydronaphthalen-1-yl)ethanol
2-(thiochroman-4-yl)ethanol:
2-(4-methylthiochroman-4-yl)ethanol
2-(4-ethylchroman-4-yl)ethanol
2-(4-propylchroman-4-yl)ethanol
   Synthesis 2: Preparation of compounds of the general formula (VI)

A. 2-(Chroman-4-yl)ethanal:

1.7 ml of DMSO in 25.0 ml of dichloromethane are added to 1 ml of oxalyl chloride in 25.0 ml of dichloromethane at −78° C. After 5 min, 1.9 g of 2-(chroman-4-yl)ethanol in 25.0 ml of dichioromethane are added dropwise at −78° C. After 15 min, 7 ml of triethylamine are added, and the mixture is slowly warmed to RT. It is washed with water, brine, 1% strength sulfuric acid and sat. sodium bicarbonate solution, dried with sodium sulfate and concentrated in vacuo. 1.81 g of the title compound are obtained as a yellow oil.

¹H NMR (CDC₁₃): δ 1.73-1.86 (m, 1H), 2.11-2.28 (m, 1H), 2.75 (dd, 1H), 2.93 (dd, 1H), 3.43-3.56 (m, lH), 4.11-4.25 (m, 2H), 6.81 (d, IH), 6.87 (t, 1H), 7.03-7.17 (m, 2H), 9.88 (1H).

B. 2-(4-Methylchroman-4-yl)ethanal:

1.0 ml of DMSO in 25.0 ml of dichloromethane are added to 0.58 ml of oxalyl chloride in 25.0 ml of dichloromethane at −78° C. After 5 min, 1.2 g of 2-(methylchroman-4-yl)ethanol in 25.0 ml of dichloromethane are added dropwise at −78° C. After 15 min, 4 ml of triethylamine are added, and the mixture is slowly warmed to room temperature. It is washed with water, brine, 1% strength sulfuric acid and sat. sodium bicarbonate solution, dried with sodium sulfate and concentrated in vacuo. Chromatography on silica gel (hexane/ethyl acetate 100:0->90:10) results in 970 mg of 2-(4-methylchroman-4-yl)ethanal as colorless oil.

¹H NMR (CDC₁₃): δ=1.48 (s, 3H), 1.92 (ddd, 1H), 2.12 (ddd, 1H), 2.59 (dd, 1H), 2.69 (dd, 1H), 4.11-4.29 (m, 2H), 6.83 (d, 1H), 6.92 (t, 1H), 7.12 (t, 1H), 7.23 (d, 1H), 9.66 (s, 1H).

C. The following can likewise be prepared by the processes described above:
2-(6-methylchroman-4-yl)ethanal
2-(7-methylchroman-4-yl)ethanal
2-(6-fluorochroman-4-yl)ethanal
2-(6-methoxychroman-4-yl)ethanal
2-(7-methoxychroman-4-yl)ethanal
2-(1,2,3,4-tetrahydronaphthalen-1-yl)ethanal
2-(2-methyl-1,2,3,4-tetrahydronaphthalen-1-yl)ethanal
2-(4-methyl-1,2,3,4-tetrahydronaphthalen-1-yl)ethanal
2-(5-methoxy-1,2,3,4-tetrahydronaphthalen-1-yl)ethanal
2-(5,7-dimethyl-1,2,3,4-tetrahydronaphthalen-1-yl)-ethanal
2-(6-methoxy-1,2,3,4-tetrahydronaphthalen-1-yl)ethanal
2-(6-fluoro-1,2,3,4-tetrahydronaphthalen-1-yl)ethanal
2-(thiochroman-4-yl)ethanal
2-(4-methylthiochroman-4-yl)ethanal
2-(4-ethylchroman-4-yl)ethanal
2-(4-propylchroman-4-yl)ethanal
   Synthesis 3: Preparation of compounds of the general formula (V)

A. 2-(Chroman-4-yl)-1-trifluoromethylethanol:

2 ml of tetrabutylammonium fluoride solution (1M in THF) are added to a solution of 1.8 g of 2-(chroman-4-yl) ethanal and 4.6 ml of trifluoromethyltrimethylsilane in 60 ml of THF at 0° C. and stirred for 1 hour. A spatula tip of solid tetrabutylammonium fluoride is added once again, and water is added. The mixture is extracted with ethyl acetate, and the organic phase is washed with brine and dried with sodium sulfate. Chromatography on silica gel (hexane/ethyl acetate 95:5->90:10) results in 2.1 g of 2-(chroman-4-yl)-1-trifluoromethylethanol as mixture of diastereomers.

¹H NMR (CDCl₃, selected signals): δ=1.76-1.97 (m, 2H), 1.98-2.34 (m, 2H), 3.08-3.25 (m, 1H), 4.03-4.35 (m, 3H), 6.88-6.95 (m, 2H), 7.06-7.21 (m, 2H).

B. 2-(4-Methylchroman-4-yl)-1-trifluoromethylethanol:

0.87 ml of tetrabutylammonium fluoride solution (1M in THF) is added to a solution of 870 mg of 2-(4-methylchroman-4-yl)ethanal and 2 ml of trifluoromethyl-trimethylsilane in 30 ml of THF at 0° C. and stirred for 1 hour. A spatula tip of solid tetrabutylammonium fluoride is added once again, and water is added. The mixture is extracted with ethyl acetate, and the organic phase is washed with brine and dried with sodium sulfate. 1.2 g of 2-(4-methylchroman-4-yl) -1-trifluoromethylethanol are obtained as brown oil (mixture of diastereomers).

¹H NMR (CDCl₃, selected signals): δ=1.34 (s, 3H) , 1.35 (s, 3H), 1.68 (ddd, 1H), 1.78 (ddd, 1H), 1.85 (dd, 1H), 1.90-2.04 (m), 2.05-2.14 (m), 2.31 (ddd, 1H), 3.73-3.83 (m, 1H), 3.97-4.22 (m), 6.77 (d, lH, diaster. A+B), 6.80-6.87 (m, 1H, diaster. A+B), 7.0-7.1 (m), 7.17-7.21 (m).

C. The following can likewise be prepared by processes described above:
2-(6-methylchroman-4-yl)-1-trifluoromethylethanol
2-(7-methylchroman-4-yl)-1-trifluoromethylethanol
2-(6-fluorochroman-4-yl)-1-trifluoromethylethanol
2-(6-methoxychroman-4-yl)-1-trifluoromethylethanol
2-(7-methoxychroman-4-yl)-1-trifluoromethylethanol
2-(1,2,3,4-tetrahydronaphthalen-1-yl)-1-trifluoromethylethanol
2-(2-methyl-1,2,3,4-tetrahydronaphthalen-1-yl)-1-trifluoromethylethanol
2-(4-methyl-1,2,3,4-tetrahydronaphthalen-1-yl)-1-trifluoromethylethanol
2-(5-methoxy-1,2,3,4-tetrahydronaphthalen-1-yl)-1-trifluoromethylethanol
2-(5,7-dimethyl-1,2,3,4-tetrahydronaphthalen-1-yl)-1-trifluoromethylethanol
2-(6-methoxy-1,2,3,4-tetrahydronaphthalen-1-yl)-1-pentafluoroethylethanol
2-(6-fluoro-1,2,3,4-tetrahydronaphthalen-1-yl)-1-trifluoromethylethanol
2-(thiochroman-4-yl)-1-trifluoromethylethanol:
2-(4-methylthiochroman-4-yl)-1-trifluoromethylethanol
2-(4-ethylchroman-4-yl)-1-trifluoromethylethanol
2-(4-propylchroman-4-yl)-i-trifluoromethylethanol
   Synthesis 4: Preparation of compounds of the general formula (IV)

A. 3-(Chroman-4-yl)-1,1,1-trifluoropropan-2-one:

54.0 g of Dess-Martin periodinane in 650 ml of dichloromethane are mixed at room temperature with a solution of 9.0 g of 2-(chroman-4-yl)-1-trifluoromethylethanol in 65 ml of dichloromethane and stirred for 10 hours. The mixture is mixed with sat. sodium bicarbonate solution, diluted with diethyl ether and stirred with sodium thiosulfate solution for 30 min. The organic phase is separated off and washed with water. It is dried with sodium sulfate and concentrated in vacuo. 7.95 g of 3-(chroman-4-yl)-1,1,1-trifluoropropan-2-one are obtained.

¹H NMR (CDCl₃): δ=1.62-1.77 (m, 1H), 2.08-2.22 (m, 1H), 2.94 (dd, 1H), 3.11 (dd, 1H), 3.39-3.50 (m, 1H), 4.02-4.20 (m, 2H), 6.75 (d, 1H), 6.81 (t, 1H ), 6.97 (d, 1H), 7.05 (t, 1H).

B. 3-(4-Methychroman-4-yl)-1,1,1-trifluoropropan-2-one:

7.8 g of Dess-Martin periodinane in 100 ml of dichloromethane are mixed at 0° C. with a solution of 1.2 g of 2-(chroman-4-yl)-1-trifluoromethylethanol in 20 ml of dichloromethane and stirred at 0° C. for 1 hour. The mixture is mixed with sat. sodium bicarbonate solution, diluted with diethyl ether and stirred with sodium thiosulfate solution for 30 min. The organic phase is separated off and washed with water. It is dried with sodium sulfate and concentrated in vacuo. 1.1 g of 3-(4-methylchroman-4-yl)-1,1,1-trifluoro-propan-2-one are obtained as brown oil.

¹H NMR (CDCl₃): δ=1.50 (s, 3H), 2.0 (ddd, 1H), 2.25 (ddd, 1H), 3.10 (s, 2H), 4.10-4.28 (tm, 2H), 6.83 (d, 1H ), 6.90 (t, 1H), 7.13 (t, 1H),.7.18 (d, 1H).

C. The following can likewise be prepared by processes described above:
2-(6-methylchroman-4-yl)-1,1,1-trifluoropropan-2-one
2-(7-methylchroman-4-yl)-1,1,1-trifluoropropan-1-one
2-(6-fluorochroman-4-yl)-1,1,1-trifluoropropan-2-one
2-(6-methoxychroman-4-yl)-1,1,1-trifluoropropan-2-one
2-(7-methoxychroman-4-yl)-1,1,1-trifluoropropan-2-one
2-(1,2,3,4-tetrahydronaphthalen-1-yl)-1,1,1-trifluoropropan-2-one
2-(2-methyl-1,2,3,4-tetrahydronaphthalen-1-yl)-1,1,1-trifluoropropan-2-one
2-(4-methyl-1,2,3,4-tetrahydronaphthalen-1-yl)-1,1,1-trifluoropropan-2-one
2-(5-methoxy-1,2,3,4-tetrahydronaphthalen-1-yl)-1,1,1-trifluoropropan-2-one
2-(5,7-dimethyl-1,2,3,4-tetrahydronaphthalen-1-yl)-1,1,1,-trifluoropropan-2-one
4-(6-methoxy-1,2,3,4-tetrahydronaphthalen-1-yl)-1,1,1,2,2-pentafluorobutan-3-one
2-(6-fluoro-1,2,3,4-tetrahydronaphthalen-1-yl)-1,1,1-trifluoropropan-2-one
2-(4-methylthiochroman-4-yl)-1,1,1-trifluoropropan-2-one
2-(4-ethylchroman-4-yl)-1,1,1-trifluoropropan-2-one
2-(4-propylchroman-4-yl)-1,1,1-trifluoropropan-2-one
   Synthesis 5: Preparation of compounds of the general formula (IIIA)

3-(Chroman-4-yl)-2-trifluoromethyl-2-trimethylsilyloxy-propionitrile

140 mg of 3-(chroman-4-yl) -1,1,1-trifluoropropan-2-one in 2.5 ml of dichloromethane are mixed with 124 mg of trimethylsilyl cyanide at room temperature and stirred for 4 hours (J. Med. Chem. 2003, (46), pp. 2494-2501). The mixture is added to water and extracted with ethyl acetate. The combined organic phases washed with brine, dried with sodium sulfate and concentrated in vacuo. 204 mg of the title compound are obtained as oil.

Synthesis 6: Preparation of compounds of the general formula (II) from compounds of the general formula (IIIA)

3-(Chroman-4-yl)-2-hydroxy-2-trifluoromethylpropion-aldhde

0.4 ml of DIBAL-H solution (1.5M in toluene) is added to 204 mg of 3-(chroman-4-yl)-2-trifluoromethyl-2-trimethylsilyloxypropionitrile in 5 ml of toluene at −70° C., and the mixture is stirred at −70° C. for 4 hours. Addition of 1 ml of ethyl acetate is followed by warming to room temperature, addition of 1M sulfuric acid and stirring for 10 hours. The mixture is diluted with water and extracted with ethyl acetate, and the combined organic phases are washed with brine, dried with sodium sulfate and concentrated in. vacuo. The remaining oil is taken up in 1 ml of THF, mixed with tetrabutylammonium fluoride solution [1M in THF] and stirred at room temperature for 6 hours. Addition of water is followed by extraction with ethyl acetate, washing with brine, drying and concentrating in vacuo. 124 mg of the title compound are obtained as an orange-colored oil.

MS (ei): MHu +=274

Synthesis 7: Preparation of compounds of the general formula (IIIB)

A. 1-(Chroman-4-yl)-2-trifluoromethyl-3-buten-2-ol:

A solution of 7.4 g of 3-(chroman-4-yl)-1,1,1-trifluoropropan-2-one in 75 ml of THF is added to 33.3 ml of vinylmagnesium bromide solution [1H in THF] at room temperature. The mixture is stirred at room temperature for 4 hours and then added to sat. ammonium chloride solution. The mixture is extracted with ethyl acetate, and the combined organic phases are washed with brine, dried with sodium sulfate and concentrated in vacuo. Chromatography on silica gel (hexane/ethyl acetate 100:0->90:10) results in 7.0 g of 1-(chroman-4-yl)-2-trifluoromethyl-3-buten-2-ol as yellow oil (mixture of diastereomers).

¹H NMR (CDCl₃, selected signals): δ=1.8-1.9 (m), 1.97 (dd, 1H), 2.01-2.37 (m), 2.93-3.03 (m, 1H), 3.03-3.12 (m, 1H), 4.05-4.27 (m), 5.53 (d, 1H), 5.58-5.70 (m, 3H), 5.97-6.17 (m, 2H), 6.73-6.94 (m, 4H), 7.02 (d, 1H), 7.04-7.13 (m, 2H), 7.21 (d, 1H).

B. 1-(4-Methylchroman-4-yl)-2-trifluoromethyl-3-buten-2-ol:

A solution of 1.1 g of 3-(4-methylchroman-4-yl)-1,1,1-trifluoropropan-2-one in 30 ml of TlF is added to 9.2 ml of vinylmagnesium bromide solution flH in THF] at room temperature. The mixture is stirred at room temperature for 5 hours and then added to sat. ammonium chloride solution. The mixture is extracted with ethyl acetate, the combined organic phases are washed with brine, dried with sodium sulfate and concentrated in vacuo. Chromatography on silica gel (heaxane/ethyl acetate 100:0->95:5) results in 930 g of 1-(4-methylchroman-4-yl)-2-trifluoromethyl-3-buten-2-ol as oil.

MS (ei): M⁺⁼286

C. The following can likewise be prepared by processes described above:
1-(6-methylchroman-4-yl)-2-trifluoromethyl-3-buten-2-ol
1-(7-methylchroman-4-yl)-2-trifluoromethyl-3-buten-2-ol
1-(6-fluorochroman-4-yl)-2-trifluoromethyl-3-buten-2-ol
1-(6-methoxychroman-4-yl)-2-trifluoromethyl-3-buten-2-ol
1-(7-methoxychroman-4-yl)-2-trifluoromethyl-3-buten-2-ol
1-(1,2,3,4-tetrahydronaphthalen-1-yl)-2-trifluoro-methyl-3-buten-2-ol
1-(2-methyl-1,2,3,4-tetrahydronaphthalen-1-yl)-2-tri-fluoromethyl-3-buten-2-ol
1-(4-methyl-1,2,3,4-tetrahydronaphthalen-1-yl)-2-tri-fluoromethyl-3-buten-2-ol
1-(5-methoxy-1,2,3,4-tetrahydronaphthalen-1-yl)-2-tri-fluoromethyl-3-buten-2-ol
1-(5,7-dimethyl-1,2,3,4-tetrahydronaphthalen-1-yl)-2-trifluoromethyl-3-buten-2-ol
1-(6-methoxy-1,2,3,4-tetrahydronaphthalen-1-yl)-2-pentafluoroethyl-3-buten-2-ol
1-(6-fluoro-1,2,3,4-tetrahydronaphthalen-1-yl)-2-tri-fluoromethyl-3-buten-2-ol
1-(4-methylthiochroman-4-yl)-2-trifluoromethyl-3-buten-2-ol
1-(4-ethylchroman-4-yl)-2-trifluoromethyl-3-buten-2-ol
1-(4-propylchroman-4-yl)-2-trifluoromethyl-3-buten-2-ol
   Synthesis 8: Preparation of comounds of the general formula (II) from compounds of the general formula (IIIB)

A. 3-(Chroman-4-yl)-2-hydroxy-2-trifluoromethylpropion-aldehyde:
Ozone is passed into a solution of 7.0 g of l-(chroman-4-yl) -2-trifluoromethyl-3-buten-2-ol in 450 ml of dichloromethane and 120 ml of methanol at −70° C. until the solution assumes a blue coloration. Argon is then passed through the solution for one minute, 3.6 ml of dimethyl sulfide are added, and the mixture is stirred at −70° C. for one hour. It is slowly warmed to room temperature and then stirred for 10 hours. The mixture is added to water and extracted with dichloromethane. The combined organic phases are dried and concentrated in vacuo. 6.6 g of 3-(chroman-4-yl)-2-hydroxy -2-trifluoromethylpropionaldehyde are obtained as yellow oil.

MS (ei): M⁺=274

B. 2-Rydroxy-3-(4-methylchroman-4-yl)-2-trifluoro-methylpropionaldehyde:

Ozone is passed into a solution of 900 mg of 1-(4-methylchroman-4-yl)-2-trifluoromethyl-3-buten-2-ol in 56 ml of dichloromethane and 14 ml of methanol at −70° C. until the solution assumes a blue coloration. Argon is then passed through the solution for one minute, 0.42 ml of dimethyl sulfide is added, and the mixture is stirred at −70° C. for one hour. It is slowly warmed to room temperature and then stirred for 10 hours. The mixture is added to water and extracted with dichloromethane. The combined organic phases are dried and concentrated in vacuo. Chromatography on'silica gel (hexane/ethyl acetate 100:0->96:4) results in 330 mg of 2-hydroxy-3-(4-methylchroman-4-yl) -2-trifluoro-methylpropionaldehyde as yellow oil (mixture of diastereomers).

¹N MR (CDCl₃, selected signals): δ=1.38 (s, 3H, diaster. A), 1.43 (s, 3H, diaster. B), 1.57-1.65 (m, 2H), 1. 81 (ddd, 1H), 2.13 (ddd, 1H), 2.37 (d, 1H, diaster. B), 2.45-2.60 (m), 2.83 (d, 1H. diaster. B), 3.69 (s, 1H, diaster. A), 3.86 (s, 1H, diaster. B), 3.97 (dd, 1H, diaster. B), 4.08-4.35 (m), 6.78-6.84 (m, 1H, diaster. A+B), 6.88-6.95 (m, 1H), 7.07-7.21 (m), 7.23-7.30 (m), 8.88 (s, lH, diaster. B), 9.56 (s, 1H, diaster. A).

C. The following can likewise be prepared by processes described above:
2-hydroxy-3-(6-methylchroman-4-yl)-2-trifluoromethyl-propionaldehyde
2-hydroxy-3-(7-methylchroman-4-yl)-2-trifluoromethyl-propionaldehyde
3-(6-fluorochroman-4-yl)-2-hydroxy-2-trifluoromethyl-propionaldehyde
2-hydroxy-3-(6-methoxychroman-4-yl)-2-trifluoromethyl-propionaldehyde
2-hydroxy-3-(7-methoxychroman-4-yl)-2-trifluoromethyl-propionaldehyde
2-hydroxy-3-(1,2,3,4-tetrahydronaphthalen-1-yl)-2-tri-fluoromethylpropionaldehyde
2-hydroxy-3-(2-methyl-1,2,3,4-tetrahydronaphthalen-1-yl)-2-trifluoromethylpropionaldehyde
2-hydroxy-3-(4-methyl-1,2,3,4-tetrahydronaphthalen-1-yl)-2-trifluoromethylpropionaldehyde
2-hydroxy-3-(5-methoxy-1,2,3,4-tetrahydronaphthalen-1-yl)-2-trifluoromethylpropionaldehyde
3-(5,⁷-dimethy-1,2,3,4-tetrahydronaphthalen-1-yl)-2-hydroxy-2-trifluoromethylpropionaldehyde
2-hydroxy-3-(6-methoxy-1,2,3,4-tetrahydronaphthalen-1-yl)-2-pentafluoroethylpropionaldehyde
3-(⁶-fluoro-1,2,3,4-tetrahydronaphthalen-1-yl)-2-hydroxy-2-trifluoromethylpropionaldehyde
2-hydroxy-3-(4-methylthiochroman-4-yl)-2-trifluoro-methylpropionaldehyde
3-(4-ethylchroman-4-yl)-2-hydroxy-2-trifluoromethyl-propionaldehyde
2-hydroxy-3-(4-propylchroman-4-yl)-2-trifluoromethyl-propionaldehyde
   Synthesis 9: Preparation of compounds of the general formula (XIII)

4-Methylenethiochroman:

79.9 ml of n-butyllithium solution [1.6M in hexanel are added dropwise to a solution of 40.9 g of methyl-triphenylphosphonium bromide in 470 ml of THF at −10° C. The mixture is stirred for a further 45 minutes and then 18.8 g of thiochroman-4-one are added. The mixture is mixed with a further 330 ml of THF and stirred at room temperature for 1 hour. The mixture is diluted with water and extracted with ethyl acetate. The combined organic phases are washed with water and brine, dried with sodium sulfate and concentrated in vacuo. Chromatography on silica gel (hexane/ethyl acetate 100:0->90:10) results in 11.7 g of 4-methylenethiochroman as oil.

¹H NMR (CDCl₃): δ=2.8-2. 9 (m, 2H), 3.03-3.13 (m, 2H) 4.98 (s, 1H ), 5.5 (6, 1H), 6.98-7.08 (m, 1H), 7.08-7.17 (m, 2H), 7.54 (d, 1H).

Synthesis 10: Preparation of compounds of the general formula (XII)

A. Ethyl 2-hydroxy-3-[(2H)thiochromen-4-yl]-2-tri-fluoromethylpropionate:

3.54 g of binaphthol are added to a solution of 1.3 ml of titanium tetraethoxide in 10 ml of toluene at room temperature and stirred for one hour. A solution of 10 g of 4-methylenethiochroman in 60 ml of toluene and 15 ml of trifluoropyruvate is added to this solution. The reaction solution is stirred at 120° C. for 3 hours and at room temperature for a further 10 hours. The reaction solution is diluted with water and ethyl acetate and filtered through kieselguhr. The organic phase is washed with brine, dried over sodium sulfate and concentrated in vacuo. Chromatography on silica gel (hexane/ethyl acetate 100:0->90:10) results in 19.8 g of ethyl 2-hydroxy-3-[(2H)thiochromen-4-yl]-2-tri-fluoromethylpropionate as brown oil.

¹H NMR (CDCl₃): δ=1.19 (t, 3H), 3.03 (d, 1H), 3.17 (dd, 1H ), 3.30 (dd, 1H), 3.81 (s, 1H), 3.87-3.97 (m, 1H), 4.09-4.18 (m, 1H ), 6.10 (t, 1H), 7.07-7.18 (m, 2H), 7.30 (d, 1H), 7.42 (d, 1H ).

B. Ethyl 3-(2H-chromen-4-yl)-2-hydroxy-2-(trifluoro-methyl)propionate

20 g (307 mmol) of zinc dust and 710 mg (2.5 mmol) of lead (II) chloride are suspended in 200 ml of THF and, at room temperature, 11.2 ml (100 mmol) of dibromo-methane are added. The mixture is stirred for a further 60 minutes, and 33 ml (33 mmol) of a 1M titanium (IV) chloride solution in dichloromethane are added dropwise over the course of 40 minutes while cooling in ice. After one hour, 4.4 g (30 mmol) of solid chroman-4-one are added in several portions at room temperature, the reaction warming to 30° C. The mixture is stirred at room temperature for a further 3 hours. It is diluted with diethyl ether and the reaction mixture is cautiously added to a mixture 4M hydrochloric acid and ice. Separation of the phases is followed by extraction with diethyl ether, washing with water, drying over sodium sulfate and removal of the solvent. The crude product is purified by column chromatography on silica gel (hexane/isopropyl ether 0-20%) to result in 2.75 g of 4-methylenechroman.

0.60 ml (0.3 mmol) of a 0.5M titanium tetraisopropoxide solution in toluene is added to 170 mg (0.60 mmol) of 1,1′-bi-2-naphthol, and the red solution is stirred at room temperature for 2 hours. 1.0 g (6.8 mmol) of 4-methylenechroman and 2.3 (13.6 mmol) of ethyl trifluoropyruvate are added, and the mixture is heated at 110° C. for 2 hours. Cooling is followed immediately by purification by column chromatography on silica gel (hexane/ethyl acetate 20%) to result in 1.15 g of ethyl 3-(2H-chromen-4-yl)-2-hydroxy-2-(trifluoromethyl)-propionate.

Synthesis 11: Preparation of compounds of the general formula (XI)

1,2-Dihydroxy- 3-((2H)thiochromen-4-yl]-2-trifluoro-methylpropane:

8.7 g of lithium aluminum hydride are added to a solution of 19.0 g of ethyl 2-hydroxy-3-[(2H) thiochromen-4-yl]-2-trifluoromethylpropionate in 500 ml of THF at 0C, and the mixture is stirred at 0° C. for 2 hours and at room temperature for 1 hour. The mixture is cautiously mixed with sat. ammonium chloride solution and extracted with ethyl acetate. The combined organic phases are washed with water and brine, dried over sodium sulfate and concentrated in vacuo. Chromatography on silica gel (hexane/ethyl acetate 100:0->80:20) results in 11 g of 1,2-dihydroxy-3-[(2H)thiochromen-4-yl]-2-trifluoromethylpropane as colorless oil.

¹H NMR (CDCl₃): δ=2.86 (d, 1H), 3.02-3.11 (m, 2H), 3.23-3.33 (m, 2H), 3.54-3.70 (m, 2H), 6.13 (t, 1H), 7.11-7.21 (m, 2H), 7.35 (d, 1H), 7.46 (d, 1H).

Synthesis 12: Preparation of compounds of the general formula (X)

1,2-Dihydroxy-3-(thiochroman-4-yl)-2-trifluoromethyl-propane:

9 g of 1,2-dihydroxy-3-[(2H)thiochromen-4-yl]-2-tri-fluoromethylpropane in 130 ml of ethanol are mixed with 9 g of Raney nickel and stirred under a hydrogen atmosphere at So bar in an autoclave for 2 hours. 9 g of Raney nickel are again added, and reaction with hydrogen at 50 bar is continued for 5 hours. The mixture is filtered with suction through kieselguhr and -concentrated in vacuo. 8.1 g of 1,2-dihydroxy-3-(thiochroman-4-yl)-2-trifluoromethylpropane are obtained as yellow oil (mixture of diastereomers).

¹H NMR (CDCl₃, selected signals): δ=1.92 (dd, 1H ), 1.95-2.07 (m), 2.18 (dd, 1H), 2.40-2.53 (m, 2H), 2.97-3.08 (m, 2H), 3.20-3.31 (m, 2H), 3.32-3.40 (m, 2H), 3.61 (t, 2H), 3.90 (d, 1H ), 3.96 (d, 1H), 7.01-7.08 (m), 7.09-7.20 (m).

Synthesis 13: Preparation of compounds of the general formula (I:) from compounds of the general formula (X)

2-Hydroxy-3-(thiochroman-4-yl)-2-trifluoromethyl-propionaldehyde:

4.6 ml of DMSO in 80 ml of dichloromethane are added to 2.4 ml of oxalyl chloride in 240 ml of dichloromethane at −78° C. After 5 min. 1.2 of 1,2-dihydroxy -3-(thiochroman-4-yl)-2-trifluoromethylpropane in 80 ml of dichloromethane are added dropwise at −78° C. After 15 min, 22.8 ml of triethylamine are added, and the mixture is slowly warmed to room temperature. It is with water and brine, dried with sodium sulfate and concentrated in vacuo. Chromatography on silica gel (hexane/ethyl acetate 100:0->85:15) results in 4.0 g of 2-hydroxy-3-(thiochroman-4-yl)-2-trifluoromethyl-propionaldehyde as yellow oil (mixture of diastereomers).

¹N MR (CDCl₃, selected signals): δ=1.75-1.93 (m) 1.95-2.03 (m), 2.12-2.28 (m), 2.60 (dd, 1H), 2.81-2.90 (m, 2H), 2.95-3.02 (m), 3.03-3.18 (m), 6.84-7.10 (m), 9.12 (s, HE, diaster. A), 9.66 (s, 1H, diaster. B).

Synthesis 14: Preparation of imines from compounds of the general formula (II)

A. 5-[3-(Chroman-4-yl)-2-hydroxy -2-trifluoromethylpropylideneamino]guinolin-2[1H]-one:

0.76 ml of titanium tetraisopropoxide is added to a solution of 600 mg of 3-(chroman-4-yl)-2-hydroxy -2-trifluoromethylpropionaldehyde and 386 mg of 5-aminoquinoiin-2[1H]-one in 22 ml of toluene, and the mixture is heated at 110° C. for 4 hours. The mixture is added to sat. sodium carbonate solution and extracted with ethyl acetate, and the combined organic phases are washed with brine, dried over sodium sulfate and concentrated in vacuo. Chromatography on silica gel (hexane 100%, dichloromethane/isopropanol 100:0->95:5) results in 275 mg of 5-(3-(chroman-4-yl)-2-hydroxy -2-trifluoromethylpropylideneamino]quinolin-2[1H]-one as beige foam (mixture of diastereomers).
¹H NMR (CDC_{13,} selected signals): δ=1.76-1.85 (m), 2.02-2.12 (m), 2.13-2.22. (m), 2.27 (d, 1H), 2.31 (d, 1H), 2.40-2.48 (mn), 2.61 (d, 1H, diaster. A+1), 2.93-3.01 (m, IH, diaster. A), 3.22-3.31 (m, 1H, diaster. B), 4.10-4.18 (m), 4.22-4.29 (m), 4.87 (bs, 1H, diaster. A), 5.02 (bs, l, diaster. B), 6.64 (d, 1H, diaster. B), 6.72 (d, IH, diaster. A), 6.73-6.84 (m), 6.88-7.00 (m), 7.06-7.17 (m), 7.24-7.31 (m), 7.37-7.42 (m), 7.45-7.54 (m), 8.06-8.18 (m), 12.43 (bs, 1H, diaster. A+B).

B. 5-[2-Hydroxy-3-(4-methylchroman-4-yl)-2-trifluoro -methylpropylideneamino]quinolin-2[1H]-one:

1.1 ml of titanium tetraisopropoxide are added to a solution of 300 mg of 3-(4-methylchroman-4-yl) -2-hydroxy-2-trifluoromethylpropionaldehyde and 160 mg of 5-aminoquinolin-2[1H]-one in 10 ml of toluene, and the mixture is heated at 110° C. for 6 hours. The mixture is added to sat. sodium bicarbonate solution and extracted with ethyl acetate, and the combined organic phases are washed with brine, dried over sodium sulfate and concentrated in vacuo. Chromatography on silica gel (hexane/ethyl acetate 100:0->0:100) results in 300 mg of 5-[2-hydroxy-3-(4-methylchroman-4-yl)-2-trifluoro -methylpropylideneaminolquinolin-2[1H]-one as foam (mixture of diastereomers).

¹H NMR (CDCl₃, selected signals): δ=1.41 (s, 3H, diaster. A), 1.46 (s, 3H, diaster. B), 1.65-1.77 (m, 1H, diaster. A), 1.81-1.94 (m, 1H, diaster. B), 2.3-2.4 (m, 1H, diaster. B), 2.43 (d, 1H, diaster. A+B), 2.61 (d, 1H, diaster. B), 2.61-2.73 (m, 2H, diaster. B), 2.87 (d, 1H, diaster. A), 3.98-4.27 (m), 4.31-4.41 (m, 1H, diaster. B), 4.82 (bs, 1H, diaster. B), 5.0 (bs, 1H, diaster. A), 5.93 (d, 1H, diaster. A), 6.49 (d, 1H, diaster. A), 6.58 (d, 1H, diaster. B), 6.72 (d, 1H, diaster. A), 6.74-6.93 (m), 7.03 (t, 1H, diaster. B), 7.2-7.34 (m), 7.38-7.50 (m, 2H, diaster. B), 8.0 (s, 1H, diaster. B), 8.05 (d, 1H, diaster. A), 8.13 (d, 1H, diaster. B), 12.65 (bs, 1H, diaster. A+B).

C. 5-[2-Hydroxy-3-(thiochroman-4-yl)-2-trifluoromethylpropylideneamino]quinolin-2[1H]-one:

2.6 ml of titanium tetraisopropoxide are added to a solution of 700 mg of 2-hydroxy-3-(thiochroman-4-yl) -2-trifluoromethylpropionaldehyde and 386 mg of 5-aminoquinolin-2[1H]-one in 20 ml of toluene, and the mixture is heated at 110° C. for 4 hours. The mixture is added to sat. sodium bicarbonate solution and extracted with ethyl acetate, and the combined organic phases are washed with brine, dried over sodium sulfate and concentrated in vacuo. Chromatography on silica gel (hexane 100%, dichloromethane/isopropanol 100:0->96:4) results in 600 mg of 5-[2-hydroxy-3-(thiochroman -4-yl)-2-trifluoromethylpropylideneamino]quinolin-2[1H]-one as foam (mixture of diastereomers).

¹H NMR (CDCl₃, selected signals): δ=1.81-2.08 (m), 2.11-2.22 (m), 2.30-2.46 (m), 2.56 (dd, 1H), 2.87-2.96 (m), 3.01-3.40 (m), 4.93 (s, 1H, diaster. A+B). 6.28 (d, 1H), 6.69 (d, 1H), 6.77 (d, lH, diaster. A+B), 6.86 (d, 1H), 6.99 (t, 1H), 7.01-7.11 (m), 7.13 (d, 1H), 7.18-7.27 (m), 7.31-7.43 (m), 7.74 (s, 1H), 8.08 (s, 1H), 8.10 (d, 1H), 8.18 (d, 1H), 12.01 (bs, 1H, diaster. A+B).

D. 5-[3-(Chroman-4-yl)-2-hydroxy-2-trifluoromethyl -propylideneamino]phthalazin-1[2H]-one:

0.76 ml of titanium tetraisopropoxide is added to a solution of 600 mg of 3-(chroman-4-yl)-2-hydroxy -2-trifluoromethylpropionaldehyde and 384 mg of 5-aminophthalazin-1 [2H]-one in 22 ml of toluene, and the mixture is heated at 110° C. for 7 hours. The mixture is added to sat. sodium bicarbonate solution and extracted with ethyl acetate, and the combined organic phases are washed with brine, dried over sodium sulfate and concentrated in vacuo. Chromatography on silica gel (hexane 100%, dichloromethane/isopropanol 100:0->95:5) results in 818 mg of 5-[3-(chroman-4-yl)-2-hydroxy-2-trifluoromethylpropylideneamino]phthalazin -1 [2H]-one as yellow foam (mixture of diastereomers). ¹NMR (CDC_{13,} selected signals): δ=1.80-1.93 (m), 2.03-2.22 (m), 2.32 (d, 1H), 2.38 (d, 1H), 2.46 (d, 1H), 2.58-2.68 (m), 2.97-3.05 (m, 1H), 3.26-3.36 (m), 4.10-4.21 (m), 4.22-4.29 (m), 4.76 (a, 1H, diaster. A), 4.89 (s, 1H, diaster. B), 6.82-6.83 (m), 6.86-7.0 (m), 7.03-7.18 (m), 7.21 (d, 1H, diaster. A), 7.70-7.81 (m), 8.06 (s, 1H, diaster. B), 8.14 (s, 1H, diaster. A), 8.33-8.55 (m), 10.47 (bs, 1H, diaster. A+B).

E. 5-[3-(Chroman-4-yl)-2-hydroxy-2-trifluoromethyl -propylideneaminol-8-fluoro-2-methylquinazoline:

0.31 ml of titanium tetraisopropoxide is added to a solution of 250 mg of 3-(chroman-4-yl)-2-hydroxy -2-trifluoromethylpropionaldehyde and 180 mg of 5-amino -8-fluoro-2-methylquinazoline in 10 ml of toluene, and the mixture is heated at 110° C. for 6 hours. The mixture is added to sat. sodium bicarbonate solution and extracted with ethyl acetate, and the combined organic phases are washed with brine, dried over sodium sulfate and concentrated in vacuo. Chromatography on silica gel dichloromethane/methanol 100:0->60:40) results in 191 mg of 5-[3-(chroman-4-yl)-2-hydroxy-2-trifluoro -methylpropylideneamino]-8-fluoro-2-methylquinazoline as orange-colored foam (mixture of diastereomers). methylquinazoline as orange-colored foam (mixture of diastereomers).

¹H NMR (CDCl₃): δ=1.80-1.92 (m, 1H), 2.02-2.22 (m), 2.33 (d, 1H), 2.38 (d, 1H), 2.46 (d, 1H ), 2.63 (dd, 1H), 2.98 (s, 3H, diaster. A+B), 3.27-3.38 (m), 4.09-4.18 (m), 4.23-4.30 (m), 4.79 (s, 1H, diaster. A), 4.91 (s, 1H, diaster. B), 6.71-6.78 (m), 6.79-6.87 (m), 6.89-6.97 (m), 7.03-7.13 (m), 7.47-7.57 (m), 8.09 (S, 1H, diaster. B), 8.18 (s, 1H, diaster. A).

F. The following can be prepared likewise:
5-[3-(chroman-4-yl)-2-hydroxy-2-trifluoromethyl-propylideneamino]-2-methylquinazoline
4-[3-(chroman-4-yl)-2-hydroxy-2-trifluoromethyl-propylideneamino]indazole
4-(3-(chroman-4-yl)-2-hydroxy-2-trifluoromethyl-propylideneamino]-1,3-dihydroindol-2-one
5-[3-(chroman-4-yl)-2-hydroxy-2-trifluoromethyl-propylideneamino]-2-methyl-iso-quinolin-1-one
5-[3-(chroman-4-yl)-2-hydroxy-2-trifluoromethyl-propylideneamino]-iso-chromen-1-one
5-(2-hydroxy-3-(4-methylchroman-4-yl)-2-trifluoro -methylpropylideneamino]phthalazin-1[2H]-one
5-[2-hydroxy-3-(4-methylchroman-4-yl)-2-trifluoro -methylpropylideneamino]-2-methylquinoline
5-[2-hydroxy-3-(4-methylchroman-4-yl)-2-trifluoro -methylpropylideneamino]-2-methylquinazoline
4-[2-hydroxy-3-(4-methylchroman-4-yl)-2-trifluoro -methylpropylideneamino]indazole
5-[2-hydroxy-3-(4-methylchrornan-4-yl)-2-trifluoro -methylpropylideneamino]-iso-chromen-1-one
5-[2-hydroxy-3-(4-methylchroman-4-yl)-2-trifluoro -methylpropylideneamino]l-iso-chromen-1-one
4-[2-hydroxy-3-(4-thiochroman-4-yl)-2-trifluoromethyl -propylideneamino]indazole
5-[2-hydroxy-3-(4-thiochroman-4-yl)-2-trifluoromethyl -propylideneamino]phthalazin-1[2H]-one
8-fluoro-5-[2-hydroxy-3-(4-thiochroman-4-yl)-2-trifluoromethylpropylideneamino]-2-methylquinazoline
4-[2-hydroxy-3-(6-methylchroman-4-yl)-2-trifluoro -methylpropylideneamino]3indazole
5-[2-hydroxy-3-(6-methylchroman-4-yl)-2-trifluoro -methylpropylideneamino]-2-methylquinoline
4-[2-hydroxy-3-(7-methylchroman-4-yl)-2-trifluoro -methylpropylideneamino]indazole
5-[2-hydroxy-3-(7-methylchroman-4-yl)-2-trifluoro -methylpropylideneamino]-2-methylquinazoline
5-[3-(6-fluorochroman-4-yl)-2-hydroxy-2-trifluoro -methylpropylideneamino]-2-methylquinazoline
5-[3-(6-fluorochroman-4-yl)-2-hydroxy-2-trifluoro -methylpropylideneamino]phthalazin-1[2H]-one
5-[3-(6-fluorochroman-4-yl)-2-hydroxy-2-trifluoro -methylpropylideneamino]-iso-chromen-1-one
5-[2-hydroxy-3-(6-methoxychroman-4-yl)-2-trifluoro -methylpropylideneamino]-2-methylquinazoline
5-[2-hydroxy-3-(7-methoxychroman-4-yl)-2-trifluoro -methylpropylideneamino]-2-methylquinazoline
5-[2-hydroxy-3-(1,2,3,4-tetrahydronaphthalen-1-yl) -2-trifluoromethylpropylideneamino]quinolin-2[1H]-one
5-[2-hydroxy-3-(1,2,3,4-tetrahydronaphthalen-1-yl) -2-trifluoromethylpropylideneamino]-2-methylquinoline
8-fluoro-5-[2-hydroxy-3-(1,2,3,4-tetrahydronaphthalen -1-yl)-2-trifluoromethylpropylideneamino]-2-methyl-quinazoline
5-[2-hydroxy-3-(2-methyl-1,2,3,4-tetrahydronaphthalen -1-yl)-2-trifluoromethylpropylideneamino]-2-methyl-quinoline
5-[2-hydroxy-3-(4-methyl-1,2,3,4-tetrahydronaphthalen -1-yl)-2-trifluoromethylpropylideneamino]-2-methyl-quinoline
5-[2-hydroxy-3-(5-methoxy-1,2,3,4-tetrahydronaphthalen -1-yl)-2-trifluoromethylpropylideneamino]-2-methyl-iso-quinolin-1-one
5-[3-(5,7-dimethyl-1,2,3,4-tetrahydronaphthalen-1-yl) -2-hydroxy-2-trifluoromethylpropylideneamino]-2-methyl-iso-quinolin-1-one
5-[2-hydroxy-3-(6-methoxy-1,2,3,4-tetrahydronaphthalen -1-yl)-2-pentafluoroethylpropylideneamino]quinolin-2[1H]-one
5-[3-(6-fluoro-1,2,3,4-tetrahydronaphthalen-1-yl) -2-hydroxy-2-trifluoromethylpropylideneamino]quinolin-2[1H]-one
5-[2-hydroxy-3-(4-methylthiochroman-4-yl)-2-trifluoro -methylpropylideneamino]quinolin-2[1H]-one
5-[2-hydroxy-3-(4-methylthiochroman-4-yl)-2-trifluoro -methylpropylideneamino]-2-methylquinazoline [symbol] [symbol]
5-[2-hydroxy-3-(4-methylthiochroman-4-yl)-2 rifluoro -methylpropylideneamino]-2-methylquinoline
5-[3-(4-ethylchroman-4-yl)-2-hydroxy-2-trifluoro -methylpropylideneamino]-2-methylquinoline
5-[3-(4-ethylchroman-4-yl)-2-hydroxy-2-trifluoro -methylpropylideneamino]quinolin-2[1H]-one
5-[3-(4-ethylchroman-4-yl)-2-hydroxy-2-trifluoro -methylpropylideneamino]phthalazin-1[2H]-one
5-[2-hydroxy-3-(4-propylchroman-4-yl)-2-trifluoro -methylpropylideneamino]quinolin-2[1H]-one
   Compounds of the general formula (I)

### Example 1

5-{[3-Chroman-4-yl)-2-hydroxy-2-trifluoromethyl -propyl]amino}quinolin-2[1H]-one:

31 mg of sodium cyanoborohydride are added to a solution of 15 mg of 5-[3-(chroman-4-yl)-2-hydroxy-2-trifluoromethylpropylideneamino]quinolin-2[1H]-one in 1.5 ml of methanol and 0.3 ml of glacial acetic acid at room temperature, and the mixture is stirred for 3.5 hours. The mixture is added to sat. sodium bicarbonate solution and extracted with ethyl acetate, and the combined organic phases are washed with brine, dried over sodium sulfate and concentrated in vacuo. Chromatography on silica gel (dichloromethane/methanol 100:0->60;40) results in 12 mg of 5-{[3-(chroman-4yl) -2-hydroxy-2-trifluoromethylpropyl]amino}quinolin -2[1H]-one as yellow foam (mixture of diastereomers).

¹H NMR (CDCl₃, selected signals): δ=2.0-2.13 (m), 2.13-2.21 (m), 3.17-3.27 (m, 1H), 3.55 (s, 1H), 3.63 (s, 1H), 4.01-4.13 (m, 2H), 6.49 (d, 1H), 6.58 (t, 1H, diaster. A+B), 6.62-6.79 (m, 3H), 6.99 (t, 1H, diaster. A+B), 7.06 (d, 1H), 7.10 (d, 1H), 7.30-7.39 (m, 1H, diaster. A+B), 8.12 (d, 1H, diaster. A+B).

### Examples 2,3,4 and 5

5-{[2-Hydroxy-3-(4-methylchroman-4-yl)-2-trifluoro -methylpropyl]amino}quinolin-2{1H}-one, diastereomer A, (+) enantiomer;
5-{[2-Hydroxy-3-(4-methylchroman-4-yl)-2-trifluoro -methylpropyl]amino}quinolin-2{1H}-one, diastereomer B, (−) enantiomer;
5-{[2-Hydroxy-3-(4-methylchroman-4-yl)-2-trifluoro -methylpropyl]amino}quinolin-2{1H}-one, diastereomer A, (−) enantiomer;
5-{[2-Hydroxy-3-(4-methylchroman-4-yl)-2-trifluoro -methylpropyl]amino}quinolin-2{1H}-one, diastereomer B, (+) enantiomer:

100 mg of sodium cyanoborohydride are added to a solution of 50 mg of 5-[2-hydroxy-3-(4-methylchroman -4-yl)-2-trifluoromethylpropylideneaminolquinolin-2[1H]-one in 4.8 ml of methanol and 1.0 ml of glacial acetic acid at room temperature, and the mixture is stirred for 3.5 hours. The mixture is added to sat. sodium bicarbonate solution and extracted with ethyl acetate, and the combined organic phases are washed with brine, dried over sodium sulfate and concentrated in vacuo. HPLC chromatography (chiral pack AD-H, hexane/ethanol 95:5->50:50) results in 1 mg of 5-{[2-hydroxy-3-(4-methylchroman-4-yl)-2-trifluoromethylpropyl]amino}-quinolin-2[1H]-one (fraction 1: diastereomer A, (+) enantiomer), 5 mg of 5-{[2-hydroxy-3-(4-methylchroman -4-yl)-2-trifluoromethylpropyl]amino}quinolin-2[1H]-one (fraction 2: diastereomer B, (−) enantiomer), 7 mg of 5-{[2-hydroxy-3-(4-methylchroman-4-yl)-2-trifluoro -methylpropyl]amino}quinolin-2[1H]-one (fraction 3: diastereomer A, (−) enantiomer), and 8 mg of 5-{[2-hydroxy-3-(4-methylchroman-4-yl)-2-trifluoromethyl -propyl]amino}quinolin-2[1H]-one (fraction 4: diastereomer B. (+) enantiomer), in each case as colorless foams.

Example 2 (fraction 1): ¹H NMR (CD₃OD): δ=1.54 (s, 3H), 1.87 (ddd, 1H ), 2.17 (d, 1H), 2.22 (d, 1H), 2.37 (ddd, 1H ), 3.42 (d, 1H ), 3.52 (d, 1H), 4.11 (ddd, 1H ), 4.22 (ddd, 1H ), 6.33 (d, 1H), 6.47 (d, 1H), 6.66 (dd, 1H), 6.78 (t, 1H), 7.0 (dt, i1), 7.27-7.33 (m, 2H), 8.0 (d, 1H ).

Example 3 (fraction 2): ¹N MR (CD₃OD): δ=1.40 (s, 3H), 1.68-1.76 (m, 1H), 2.15 (d, 1H), 2.53 (d, 1H), 2.85 (d, 1H), 2.87-2.97 (m, 1H), 3.06 (d, 1H), 4.02-4.11 (m, li), 4.20-4.27 (m, 1H), 5.74 (d, 1H), 6.44 (d, 1H), 6.59 (d, 1H), 6.7 (d, 1H), 6.88 (t, 1H), 7.04 (t, 1H), 7.15 (t, 1H), 7.36 (d, 1H ), 7.96 (d, 1H).

Example 4 (fraction 3): 2H NMR (CD30D): δ=1.54 (s, 3H), 1.87 (ddd, 1H), 2.17 (d, 1H ), 2.22 (d, 1H), 2.37 (ddd, 1H), 3.42 (d, 1H), 3.52 (d, 1H), 4.11 (ddd, 1H), 4.22 (ddd, 1H), 6.33 (d, 1H), 6.47 (d, 1H), 6.66 (dd, 1H), 6.78 (t, 1H), 7.0 (dt, 1H), 7.27-7.33 (m, 2H), 8.0 (d, 1H ).

Example 5 (fraction 4): ¹H NMR (CD₃OD): δ=1.40 (s, 3H), 1.68-1.76 (m, 1H ), 2.15 (d, 1H), 2.53 (d, 1H), 2.85 (d, 1H), 2.87-2.97 (m, 1H), 3.06 (d, 1H), 4.02-4.11 (m, 1H), 4.20-4.27 (m, 1H), 5.74 (d, 1H), 6.44 (d, 1H ), 6.59 (d, 1H ), 6.7 (d, 1H), 6.88 (t, 1H), 7.04 (t, 1H), 7.15 (t, 1H), 7.36 (d, 1H), 7.96 (d, 1H).

### Example 6

5-{[2-Hydroxy-3-(thiochroman-4-yl)-2-trifluoromethyl -propyl]amino}quinolin-2[1H]-one: 100 mg of sodium cyanoborohydride are added to a solution of 50 mg of 5-[2-hydroxy-3-(thiochroman-4-yl) -2-trifluoromethylpropylideneamino]quinolin-2[1H]-one in 4.8 ml of methanol and 1.0 ml of glacial acetic acid at room temperature, and the mixture is stirred for 1 hour. The mixture is added to sat. sodium bicarbonate solution and extracted with ethyl acetate, and the combined organic phases are washed with brine, dried over sodium sulfate and concentrated in vacuo. 44 mg of 5-{[2-hydroxy-3-(thiochroman-4-yl)-2-trifluoromethyl -propyl]amino}quinolin-2[1H]-one are obtained as a yellow foam (mixture of diastereomers).

¹H NMR (CDCl₃, selected signals): δ=1.80-1.92 (m), 2.07-2.12 (m), 2.18 (dd, lI), 2.30 (dd, 1H ), 2.32-2.40 (m, 1H ), 2.88-2.95 (m), 3.05-3.18 (m), 3.20-3.31 (m), 3.31.-3.38 (m), 3.42 (dd, JH), 4.11-4.23 (m), 6.17 (d, 1H ), 6.22 (d, 1H ), 6.44 (t, 1H ), 6.54 (d, 1H), 6.60 (d, 1H), 6.85-7.20 (m), 7.51-7.57 (m), 11.25-11.44 (bd, 1H, diaster. A+B).

### Example 7

3-(Chroman-4-yl)-1-[(2-methylquinolin-5-yl)amino]-2-(trifluoromethyl)propan-2-ol

A. 1.15 g of ethyl 3-(2H-chromen-4-yl)-2-hydroxy-2-(trifluoromethyl)propionate are taken up in 30 ml of diethyl ether and cooled to −5° C. 275 mg (7.2 mmol) of solid lithium aluminum hydride are added in portions over the course of 10 minutes. The mixture is stirred at this temperature for 2 hours and poured into saturated ammonium chloride solution. The suspension is filtered through Celite, washing thoroughly with ethyl acetate. The phases of the filtrate are separated, and ethyl acetate extraction is repeated. Washing with saturated sodium chloride solution and drying over sodium sulfate are followed by removal of the solvent in vacuo. Chromatographic purification on silica gel (hexane/ethyl acetate 25-33%) affords 620 mg of 3-(2H -chromen-4-yl)-2-hydroxy-2-(trifluoromethyl) propanal.

¹H NMR (300 MHz, CDCl₃); δ=3.06 (d, 1H), 3.26 (d, 1H), 3.89 (a, 1H), 4.67 (dd, 1H), 4.70 (dd, 1H), 5.74 (t, 1H ), 6.80 (d, 1H), 6.93 (t, 1H ) , 7.15 (t, 1H), 7.21 (d, 1H), 9.63 (s, 1H) .

B. 0.4 ml (2.0 mmol) of titanium tetraethoxide is added to 245 mg (0.9 mmol) of 3-(2H-chromen-4-yl)-2-hydroxy -2-(trifluoromethyl)propanal and 150 mg (0.95 mol) of 5-amino-8-methylquinoline in 10 ml of toluene, and the mixture is heated at 100° C. for 2 hours. After cooling, it is poured into water and stirred vigorously. The suspension is filtered through Celite, washing thoroughly with ethyl acetate. The phases of the filtrate are separated, and ethyl acetate extraction is repeated. Drying over sodium sulfate is followed by removal of the solvent in vacuc. Purification by chromatography on silica gel (hexane/ethyl acetate 50%) results in 180 mg of 3-(2H-chromen-4-yl)-1-[(2-methylquinolin-5-yl)imino]-2-(trifluoromethyl)propan-2-ol. 90 mg of the imine obtained in this way are taken up in 4 ml of methanol and cooled to 0° C. 25 mg (0.66 mmol) of solid sodium borohydride are added, followed after 2 hours by dropwise addition pouring into saturated ammonium chloride solution and vigorous stirring. Extraction with ethyl acetate several times, washing with saturated sodium chloride solution and drying over sodium sulfate are followed by removal of the solvent in vacuo. Separation by column chromatography on silica gel (hexane/ethyl acetate 50%) affords 80 mg of 3-(2H-chromen-4-yl)-1-[(2-methylquinolin-5-yl)amino]-2-(trifluoromethyl)propan-2-ol.

C. 30 mg of 3-(2H-chromen-4-yl)-1-[(2-methylquinolin-5-yl)amino]-2-(trifluoromethyl)propan-2-ol are taken up in 8 ml of methanol and, after addition of 10 mg of palladium on carbon (10%), the suspension is shaken under hydrogen at atmospheric pressure for 2 hours. The mixture is filtered through Celite, washing thoroughly with methanol. The solvent is removed in vacuo and, after purification by chromatography on silica gel (hexane/ethyl acetate 50%), 12 mg of the title compound are obtained as mixture of diastereomers.

¹NMR (300 MHz, CDCl₃); δ=2.05-2.37 (m, 4H), 2.74 (a, 3H), 3.24-3.32 (m, 1H), 3.51 (dd, 0.5H), 3.50 (dd, 1H), 3.61-3.72 (m, 1.5H), 4.21 (m, 2H), 4.45 (br, 1ii) 6.70 (dd, 0.5H), 6.75 (dd, 0.5H), 6.78-6.88 (m, 2H), 7.08-7.13 (m, 1.5HR), 7.18 (d, 0.5H), 7.27 (d, 1H), 7.53-7.56 (m, 2H), 8.04 (d, 0.5H), 8.06 (d, 0.5H).

The following can likewise be prepared by methods described above:
3-(chroman-4-yl)-1-(2-methylquinolin-5-ylamino)-2-tri -fluoromethylpropan-2-ol
5-{[3-(chroman-4-yl)-2-hydroxy-2-trifluoromethyl -propyl]amino}phthalazin-1[2H]-one
5-{[3-(chroman-4-yl)-2-hydroxy-2-trifluoromethyl -propyl]amino}-8-fluoro-2-methylquinazoline
5-{[3-(chroman-4-yl)-2-hydroxy-2-trifluoromethyl -propyl]amino}-2-methylquinazoline
4-{[3-(chroman-4-yl)-2-hydroxy-2-trifluoromethyl -propyl]amino)indazole
4-([3-(chroman-4-yl)-2-hydroxy-2-trifluoromethyl -propyl]amino}-1,3-dihydroindol-2-one
5{([3-(chroman-4-yl)-2-hydroxy-2-trifluoromethyl -propyl]amino}-2-methyl-iso-quinolin-1-one
5-{[3-(chroman-4-yl)-2-hydroxy-2-trifluoromethyl -propyl]amino}-iso-cchromen-1-one
5-{[2-hydroxy-3-(4-methylchroman-4-yl)-2-trifluoro -methylpropyl]amino)phthalazin-1[2H]-one
5-{[2-hydroxy-3-(4-methylchroman-4-yl)-2-trifluoro -methylpropyl]amino}-2-methylquinoline
5-([2-hydroxy-3-(4-methylchroman-4-yl)-2-trifluoro -methylpropyl]amino)-2-methylquinazoline
4-{[2-hydroxy-3-(4-methylchroman-4-yl)-2-trifluoro -methylpropyl]amino}indazole
5-{[2-hydroxy-3-(4-methylchroman-4-yl)-2-trifluoro -methylpropyl]amino}-iso-chromen-1-one
5-{[2-hydroxy-3-(4-methylchroman-4-yl)-2-trifluoro -methylpropyl]amino}-iso-chromen-1-one
4-{[2-hydroxy-3-(4-thiochroman-4-yl)-2-trifluoromethyl -propyl]amino}indazole
5-{[2-hydroxy-3-(4-thiochroman-4-yl)-2-trifluoromethyl -propyl]amino}phthalazin-1[2H]-one
8-fluoro-5-{[2-hydroxy-3-(4-thiochroman-4-yl)-2-tri -fluoromethylpropyl]amino}-2-methylquinazoline
4-{[2-hydroxy-3-(6-methylchroman-4-yl)-2-trifluoro -methylpropyl]amino}indazole
5-{[2-hydroxy-3-(G-methylchroman-4-yl)-2-trifluoro -methylpropyl]amino}-2-methylquinoline
4-{[2-hydroxy-3-(7-methylchroman-4-yl)-2-trifluoro -methylpropyl]amino}indazole
5-{[2-hydroxy-3-(7-methylchroman-4-yl)-2-trifluoro -methylpropyl]amino}-2-methylquinazoline
5-{[3-(6-fluorochroman-4-yl)-2-hydroxy-2-trifluoro -methylpropyl]amino}-2-methylquinazoline
5-{[3-(6-fluorochroman-4-yl)-2-hydroxy-2-trifluoro -methylpropyl]amino}phthalazin-1[2H]-one
5-{[3-(6-fluorochroman-4-yl)-2-hydroxy-2-trifluoro -methylpropyl]amino}-iso-chromen-1-one
5-{[2-hydroxy-3-(6-methoxychroman-4-yl)-2-trifluoro -methylpropyl]amino}-2-methylquinazoline
5-{[2-hydroxy-3-(7-methoxychroman-4-yl)-2-trifluoro -methylpropyl]amino}-2-methylquinazoline
5-{[2-hydroxy-3-(1,2,3,4-tetrahydronaphthalen-1-yl) -2-trifluoromethylpropyl]amino}quinolin-2[1]-one
5-{[2-hydroxy-3-(1,2,3,4-tetrahydronaphthalen-1-yl) -2-trifluoromethylpropyl]amino}-2-methylquinoline
8-fluoro-5-{[2-hydroxy-3-(1,2,3,4-tetrahydronaphthalen -1-yl)-2-trifluoromethylpropyl]amino}-2-methyl-quinazoline
5-{[2-hydroxy-3-(2-methyl-1,2,3,4-tetrahydronaphthalen -1-yl)-2-trifluoromethylpropyl]amino}-2-methylquinoline
5-{[2-hydroxy-3-(4-methyl-1,2,3,4-tetrahydronaphthalen -1-yl)-2-trifluoromethylpropyl]amino}-2-methylquinoline
5-{[2-hydroxy-3-(5-methoxy-1,2,3,4-tetrahydro -naphthalen-1-yl)-2-trifluoromethylpropyl]amino}-2-methyl-iso-quinolin-1-one
5-{[3-(5,7-dimethyl-1,2,3,4-tetrahydronaphthalen-1-yl) -2-hydroxy-2-trifluoromethylpropyl]amino}-2-methyl-iso-quinolin-1-one
5-{[2-hydroxy-3-(6-methoxy-1,2,3,4-tetrahydro -naphthalen-1-yl)-2-pentafluoroethylpropyl]-amino}quinolin-2[1H]-one
5-{[3-(6-fluoro-1,2,3,4-tetrahydronaphthalen-1-yl) -2-hydroxy-2-trifluoromethylpropyl]amino}quinolin-2[1H]-one
5-{[2-hydroxy-3-(4-methylthiochroman-4-yl)-2-trifluoro -methylpropyl]amino}quinolin-2[1H]-one
5-{[2-hydroxy-3-(4-methylthiochroman-4-yl)-2-trifluoro -methylpropyl]amino}-2-methylquinazoline
5-{[2-hydroxy-3-(4-methylthiochroman-4-yl)-2-trifluoro -methylpropyl]amino}-2-methylquinoline
5-{[3-(4-ethylchroman-4-yl)-2-hydroxy-2-trifluoro -methylpropyl]amino}-2-methylquinoline
5-{[3-(4-ethylchroman-4-yl)-2-hydroxy-2-trifluoro -methylpropyl]amino}quinolin-2[1H]-one
5-{[3-(4-ethylchroman-4-yl)-2-hydroxy-2-trifluoro -methylpropyl]amino}phthalazin-1[2H]-one
5-{[2-hydroxy-3-(4-propylchroman-4-yl)-2-trifluoromethylpropyl]amino}quinolin-2[1H]-one

1. A compound of the general formula (I) 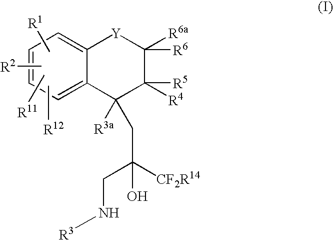
in which
R¹ and R² are independently of one another a hydrogen atom, a hydroxy group, a halogen atom, an optionally substituted (C₁₋₁₀)-alkyl group, a (C₁₋₁₀)-alkoxy group, a (C₁₋₁₀)-alkylthio group, a (C₁₋₅)-perfluoroalkyl group, a cyano group, a nitro group, or an -NR⁹R^{9a} group,
or R¹ and R² together form a group selected from the groups —O—(CH₂)ₙ—O—, —0—(CH₂)ₙ-CH₂—, —O—CH═CH—, —(CH₂)ₙ₊₂—, —NH—(CH₂)ₙ₊₁—, —N(C₁₋₃-alkyl) —(CH₂)ₙ₊₁—and —NH—N═CH—,
where n is 1 or 2, and the terminal oxygen atoms and/or carbon atoms and/or nitrogen atoms are linked to directly adjacent ring carbon atoms,
R¹¹ is a hydrogen atom, a hydroxy group, a halogen atom, a cyano group, an optionally substituted (C₁-C₁₀)-alkyl group, a (C₁-C₁₀ )-alkoxy group, a (C₁-C₁₀)-alkylthio group, or a (C₁-C₅)—perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a (C₁-C₁₀)-alkyl group which is optionally substituted by 1 to 3 hydroxy groups, 1 to 3 halogen atoms, and/or 1 to 3 (C₁-C₅)-alkoxy groups, an optionally substituted (C₃-C₇)-cycloalkyl group, an optionally substituted heterocyclyl group, an optionally substituted aryl group, or a mono- or bicyclic heteroaryl group which is optionally substituted by one or more groups which are selected independently of one another from
(C₁-C₅)-alkyl groups which themselves may optionally be substituted by 1 to 3 hydroxy or 1 to 3 —COOR¹³ groups,
(C₁-C₅) -alkoxy groups, halogen atoms, hydroxy groups, —NR⁹R^{9a} groups, (C₁-C₅)-perfluoroalkyl groups, nitro groups, thiol groups, sulfoxyl groups, sulfonic acid groups, sulfonamide groups, sulfonimine groups, cyano groups or —(CO)—(C₁-C₅)-alkyl groups, and
exo methylene groups and optionally comprises 1 to 4 nitrogen atoms and/or 1 to 2 oxygen atoms and/or 1 to 2 sulfur atoms and/or 1 to 2 keto groups, this group being linked by any position to the nitrogen atom and possibly being optionally hydrogenated at one or more positions,
R^{3a} is a hydrogen atom, a cyano group or an optionally substituted (C₃-C₅)-alkyl group;
R⁴, R⁵, R⁶ and R^{6a} are independently of one another a hydrogen atom, a halogen atom, a hydroxy group, an —NR⁹R^{9a} group, an optionally substituted (C₁-C₁₀)-alkyl group, a (C₁-C₁₀)-alkoxy group or a (C₁-C₁₀)-alkylthio group,
R⁹ and R^{9a} are independently of one another a hydrogen atom, a (C₁-C₅)-alkyl group or a —(CO)—(C₁-C₅)-alkyl group,
R¹⁰ is a (C₁-C₁₀)-alkyl group or a —(CO)—(C₁-C₁₀)-alkyl group,
R¹³ is a hydrogen atom or a (C₁-C₅)-alkyl group,
R¹⁴ is a hydrogen atom, a fluorine atom or a partly or completely fluorinated (C₁-C₅)-alkyl group, and
Y is a methylene group, an oxygen atom, a sulfur atom, an —S(O)ₙ group (where n=1 or 2), an —S(O)(NR¹³) group, an —NH group or an —NR¹⁰ group;
in the form of any stereoisomer or of a mixture of stereoisomers; or as pharmacologically acceptable salt or derivative.

2. A compound as claimed in claim 1, where
Y is an oxygen atom, a sulfur atom or a methylene group.

3. A compound as claimed in claim 1 or 2, where
R³ is an optionally substituted aryl or heteroaryl group, preferably selected from the group consisting of naphthyl, phthalidyl, isoindolyl, dihydroindolyl, dihydroisoindolyl, dihydroisoquinolinyl, thio-phthalidyl, benzofuranyl, benzoxazinonyl, phthal-azinonyl, quinolinyl, isoquinolinyl, quinolonyl, isoquinolonyl, chromanyl, isochromanyl, indazolyl, benzothiazolyl, quinazolinyl, quinoxalinyl, cinnolinyl, phthalazinyl, 1,7- or 1,8-naphthyridinyl, pyrazolo-[1,5-a]pyridyl, dihydroindolonyl, dihydroisoindolonyl, benzimidazole and indolyl group.

4. A compound as claimed in any of the preceding claims, where
R^{3a} is a hydrogen atom or a (C₁-C₅)-alkyl group.

5. A compound as claimed in any of the preceding claims, where
R⁴, R⁵, R⁶ and R^{6a} are independently of one another a hydrogen atom, a halogen atom or an optionally substituted (C₁-C₁₀)-alkyl group.

6. A compound as claimed in any of the preceding claims, where
R¹⁴ is a fluorine atom or a trifluoromethyl group.

7. A compound as claimed in any of the preceding claims for manufacturing a medicament.

8. The use of a compound as claimed in any of claims 1 to 6 for manufacturing a pharmaceutical composition for the treatment or prevention of inflammatory processes.

9. A method for the treatment or prevention of inflammatory processes in a patient, characterized in that a pharmaceutically effective amount of a compound of the formula (I) as claimed in any of claims 1 to 6 is administered to a patient requiring such a treatment or prevention.

10. A pharmaceutical product comprising at least one compound as claimed in any of claims 1 to 6 and one or more pharmaceutically acceptable carriers and/or excipients.

11. A process for preparing compounds as claimed in any of claims 1 to 6, characterized in that a compound of the formula (II) 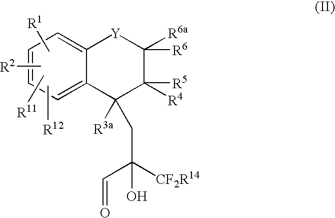
is reacted with an amine of the formula R³—NH₂, and the resulting imine is reduced to a compound of the formula (I), where the substituents R¹ to R¹⁴ and Y have the meanings specified in claims 1 to 6.

12. A compound of the formula (II), 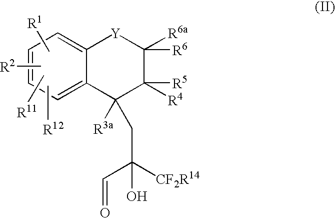
where the substituents R¹ to R¹⁴ and Y have the meanings specified in claims 1 to 6.

13. The use of compounds of the general formula (II) as defined in claim 12 for preparing compounds of the general formula (I) as claimed in any of claims 1 to 6.
